(19) United States
(12) Patent Application Publication
(10) Pub. No.: US 2024/0167086 A1
(43) Pub. Date: May 23, 2024

(54) NUCLEOSIDE-5'-OLIGOPHOSPHATES HAVING A CATIONICALLY-MODIFIED NUCELOBASE

(71) Applicant: Roche Sequencing Solutions, Inc., Pleasanton, CA (US)

(72) Inventors: Peter Crisalli, Mountain View, CA (US); Dieter Heindl, Munich (DE); Omid Khakshoor, Lathrop, CA (US); Hannes Kuchelmeister, Munich (DE); Martin Mex, Munich (DE); Meng C. Taing, Hayward, CA (US)

(21) Appl. No.: 18/542,500

(22) Filed: Dec. 15, 2023

Related U.S. Application Data
(63) Continuation of application No. PCT/EP2022/066263, filed on Jun. 15, 2022.
(60) Provisional application No. 63/202,626, filed on Jun. 17, 2021.

(51) Int. Cl. C12Q 1/6869; B82Y 5/00; C07H 19/10; C07H 19/20

(52) U.S. Cl. CPC C12Q 1/6869; B82Y 5/00; C07H 19/10; C07H 19/20

(57) ABSTRACT

Disclosed herein are base-modified nucleoside-5′-oligophosphates (bm-N5OP) that include a positively charged moiety at least at one position of the base, compositions comprising the same, compositions made from the same, methods of making the same, and methods of using the same. The bm-N5OP disclosed herein are useful, for example, as tagged nucleotides for use in nanoSBS methods and for generating primers and/or templates for use in nanoSBS methods. When incorporated into a polynucleotide, the disclosed bm-N5OPs can neutralize at least a portion of the negative charge of the overall polynucleotide molecule.

### CROSS-REFERENCE TO RELATED APPLICATION(S)

This patent application is a continuation of International Patent Application No. PCT/EP2022/066263, filed Jun. 15, 2022, which claims priority to and the benefit of U.S. Provisional Application No. 63/202,626, Jun. 17, 2021. Each of the above patent applications is incorporated herein by reference as if set forth in its entirety.

### STATEMENT REGARDING SEQUENCE LISTING

The Sequence Listing associated with this application is provided in ST.26 XML format in lieu of a paper copy, and is hereby incorporated by reference in its entirety into the specification. The name of the XML file containing the Sequence Listing is P36939-US-1_Sequence_Listing.xml. The ST.26 XML file is 6,000 bytes, was created on Dec. 14, 2023, and is being submitted electronically via Patent Center.

### BACKGROUND OF THE INVENTION

### A. Technical Field

Modified nucleoside-5′-oligophosphates and uses thereof for amplifying and/or sequencing nucleic acids.

### B. Description of Related Art

Modified canonical nucleotides have found many uses. For example, Xu et al. review fluorescence-enhancing modifications to canonical purines and pyrimidines, including purine or pyrimidine ring structure modifications; extended fluorescent scaffolds via conjugated linkers; purine or pyrimidine substituent modifications; and purine and pyrimidine ring fusions. These structures have been used, for example, in single nucleotide polymorphism detection, microenvironment monitoring, structural and morphological measurement, and polymerase activity testing. Hocek and Fojta disclose various methods for adding redox active moieties to canonical nucleobases. Prober et al. disclose dideoxynucleotides labelled with a succinylfluorescein dye for use as chain terminators in dideoxy DNA sequencing protocols. The dye is attached to the nucleobase via a linker at the 5 position in pyrimidines and at the 7 position in 7-deazapurines.

One particular application of modified nucleotides is nanopore-based sequencing-by-synthesis (nanoSBS). In nanoSBS methods, polymer-tagged nucleotides are polymerized in proximity to the entrance to the nanopore. As each tagged nucleotide is incorporated into the growing amplicon, the polymer tag enters into the nanopore and changes at least one electrochemical characteristic of the nanopore (such as current flow or resistance of the pore). By equipping each canonical nucleotide with a tag that generates a unique electrochemical signature, the sequence of nucleotides incorporated into the amplicon can be identified. Exemplary tag-based nanoSBS approaches and materials for performing such methods are described at, for example, WO 2012-083249, WO 2013/154999, US 2014/0309144, U.S. Pat. No. 9,017,937, WO 2015/148402, WO 2016/069806, WO 2016/144973, US 2013/0244340, US 2013/0264207, US 2014/0134616 US 2016/0222363, US 2016/0333327, WO 2017/050728, WO 2017/184866, WO 2017/050722, US 2017/0267983, US 2018/0245147, US 2018/0094249, WO 2018/002125, and Kumar. US 2013-0264207 discloses tagged nucleotides, including nucleotides having tags positioned at the phosphate, the sugar moiety, or at the base of the nucleotide. In each of these cases, the tag is intended to be inserted into the pore and cleaved from the nucleotide upon or shortly after incorporation into a growing amplicon.

One issue with nanoSBS methods is that many common nanopores are positively charged, which tends to attract negatively charged nucleic acids into the pore. This can result in increased background on the nanopore system and a loss of active sites at which sequencing occurs. There remains a need to identify methods of mitigating these issues.

### SUMMARY OF THE INVENTION

Disclosed herein are base-modified nucleoside-5′-oligophosphates (bm-N5OP) that include a positively charged moiety at least at one position of the base, compositions comprising the same, compositions made from the same, methods of making the same, and methods of using the same. The bm-N5OP disclosed herein are useful, for example, as tagged nucleotides for use in nanoSBS methods and for generating primers and/or templates for use in nanoSBS methods.

In an exemplary embodiment, the bm-N5OP (or a salt thereof) is provided, the bm-N5OP having a structure according to Formula 1:

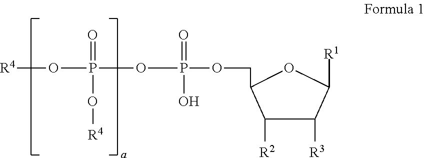

wherein:
R¹ is selected from the group consisting of:

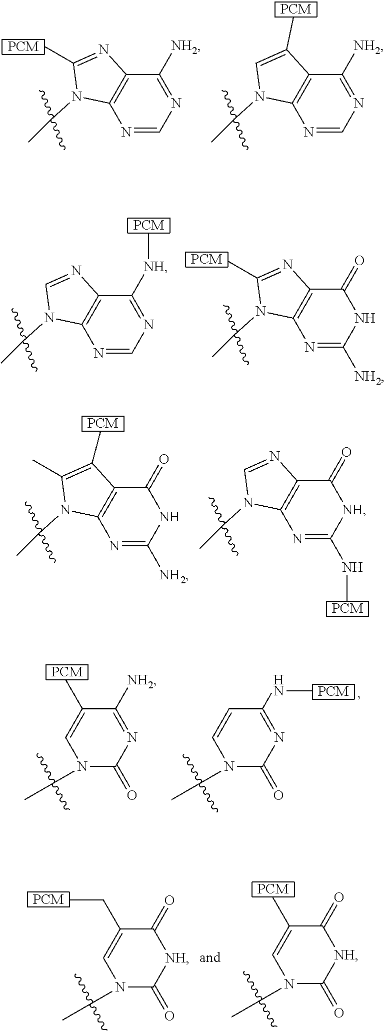

wherein PCM is a moiety having a net-positive charge at 25° C. when in a reference solution buffered at pH 7-8 and comprising 450 mM potassium acetate; R² is selected from the group consisting of H and OH; R³ is selected from the group consisting of H, OH, F, and —O—CH₃; R⁴ is H or a nanopore-detectable tag construct, with the proviso that not more than one instance of R⁴ is the nanopore-detectable tag construct; and a is from 2 to 12.

Exemplary PCM moieties include those according to Formula 2:

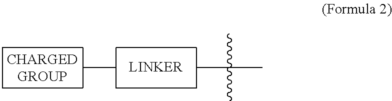

wherein CHARGED GROUP is a chemical group that has a net positive charge (including, but not limited to, primary amines, secondary amines, tertiary amines, quaternary amines, guanidinium groups, phosphonium groups, and a heteroaromatic rings) and LINKER is a chemical group covalently linking CHARGED GROUP to the nucleobase (including but not limited to alkanes, alkenes, alkynes, aryl groups, heteroaryl groups, amides, ethers, and polyethers). Exemplary PCM structures within the scope of Formula 2 include, but not limited to, Formulas 2a-2h:

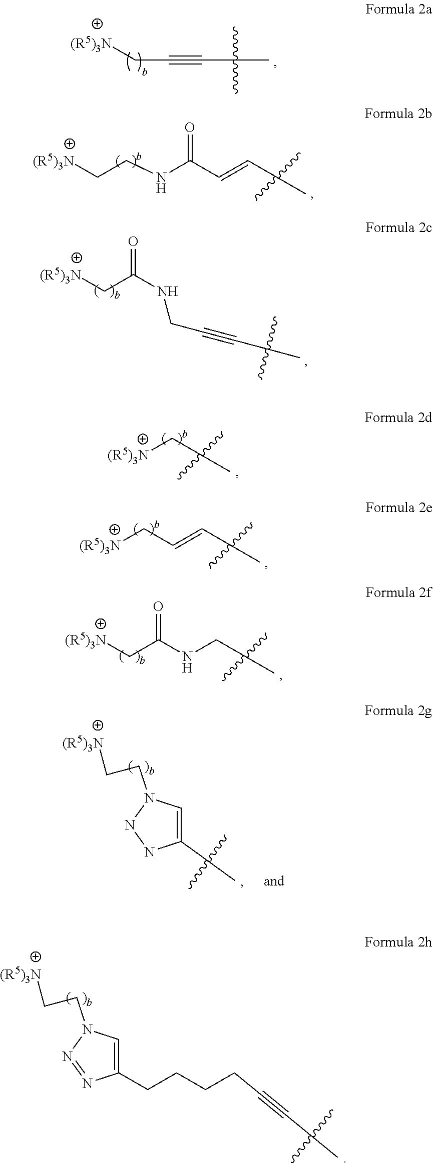

wherein R⁵ is selected from the group consisting of H, F, Cl, Br, alkyl, and alkyl halide, and b is from 1 to 12.

Also disclosed herein are sets of nucleotides including 1 or more of the bm-N5OPs disclosed herein. Exemplary sets of bm-N5OPs include those disclosed at Tables 1 and 2.

Also disclosed herein are nucleic acids comprising 1 or more base-modified nucleobases disclosed herein, including, for example, template nucleic acids and/or primer nucleic acids useful for template-dependent amplification reactions.

Also disclosed herein are methods of sequencing nucleic acids using the bm-N5OPs, sets of N5OPs, and nucleic acids disclosed herein, as well as systems for performing the same.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 illustrates an exemplary nanopore sequencing complex.

FIG. 2 is a top view of an exemplary nanopore sensor chip.

FIG. 3 illustrates an exemplary nanopore cell comprising a nanopore sequencing complex.

FIG. 4 illustrates an exemplary embodiment of an active sequencing complex performing a tag-based SBS nucleic acid sequencing method.

FIG. 5 illustrates an exemplary SBS sequencing run showing the problem of template/primer insertion.

FIG. 6A illustrates an exemplary scheme for synthesizing a bm-dC5OP.

FIG. 6B illustrates an exemplary scheme for tagging the bm-dC5OP illustrated in FIG. 6A.

FIG. 7 is a bar graph illustrating a reduction in the fraction of threaded pores when using a bm-dC5OP in a nanoSBS sequencing reaction. A is the fraction of threaded pores observed when the set of dN5OPs includes bm-dC5OP, while B is the fraction of threaded pores observed using only native dN5OPs.

FIG. 8A is a heat map of threaded pores on a chip during the first pass of a sequencing run. “Template 1” and “Template 2” refer to the different strands of the template being used. The X-Axis indicates the position along the template nucleic acid at which a recording is made. Each tick along the Y-Axis is a recording in an individual cell. The colors of the ticks indicate the template background intensity level, from low (red) to high (purple), with lower intensity indicating less background due to template threading and higher intensity indicating higher background due to template threading. The heat maps labelled with “A” were generated from sequencing runs using an N5OP set that includes only native dN5OPs. The heat maps labelled with “B” were generated from sequencing runs using an N5OP set that includes a bm-dC5OP.

FIG. 8B is a heat map of threaded pores on a chip during 5 passes of a sequencing run. “Template 1” and “Template 2” refer to the different strands of the template being used. The X-Axis indicates the position along the template nucleic acid at which a recording is made. Each tick along the Y-Axis is an individual cell, while the numbers on the Y-axis indicate how many laps around the template have been completed. The colors of the ticks indicate the template background intensity level, from low (red) to high (purple), with lower intensity indicating less background due to template threading and higher intensity indicating higher background due to template threading. The heat maps labelled with “A” were generated from sequencing runs using an N5OP set that includes only native N5OPs. The heat maps labelled with “B” were generated from sequencing runs using an N5OP set that includes a bm-dC5OP.

FIG. 9 is a chart of A-deletions and C-deletions detected using native N5OPs (A) versus a set of dN5OPs including a bm-dC5OP (B). The X-axis is the position along the template at which a capture event is recorded and each tick along the Y-axis is a C- or an A-non-cognate deletion recorded at an individual cell of the chip. Black “V” marks at the top of each trace indicate the start of a pass along the template.

### DETAILED DESCRIPTION OF THE INVENTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding (i) either or (ii) both of those included limits are also included in the invention. For example “1 to 50” includes “2 to 25”, “5 to 20”, “25 to 50”, “1 to 10”, etc.

It is to be understood that both the foregoing general description, including the drawings, and the following detailed description are exemplary and explanatory only and are not restrictive of this disclosure.

### A. DEFINITIONS

The technical and scientific terms used in the descriptions herein will have the meanings commonly understood by one of ordinary skill in the art, unless specifically defined otherwise. Accordingly, the following terms are intended to have the following meanings.

“Nucleic acid,” as used herein, refers to a molecule of one or more nucleic acid subunits which comprise one of the nucleobases, adenine (A), cytosine (C), guanine (G), thymine (T), and uracil (U), or variants thereof. Nucleic acid can refer to a polymer of nucleotides (e.g., dAMP, dCMP, dGMP, dT/dUMP), also referred to as a polynucleotide or oligonucleotide, and includes DNA, RNA, in both single and double-stranded form, and hybrids thereof.

“Nucleic acid template,” as used herein, refers to a nucleic acid or portion thereof that is capable of use as a guide for polymerase catalyzed replication. A nucleic acid molecule can include multiple templates along its length or, alternatively, only a single template may be used in a particular embodiment herein. A nucleic acid template can also function as a guide for ligase-catalyzed primer extension.

“Nucleotide,” as used herein, refers to a nucleoside-5′-oligophosphate compound, or structural analog of a nucleoside-5′-oligophosphate, which is capable of acting as a substrate or inhibitor of a nucleic acid polymerase. Exemplary nucleotides include, but are not limited to, nucleoside-5′-triphosphates (e.g., dATP, dCTP, dGTP, dTTP, and dUTP); nucleosides (e.g., dA, dC, dG, dT, and dU) with 5′-oligophosphate chains of 4 or more phosphates in length (e.g., 5′-tetraphosphosphate, 5′-pentaphosphosphate, 5′-hexaphosphosphate, 5′-heptaphosphosphate, 5′-octaphosphosphate); and structural analogs of nucleoside-5′-triphosphates that can have a modified base moiety (e.g., a substituted purine or pyrimidine base), a modified sugar moiety (e.g., an O-alkylated sugar), and/or a modified oligophosphate moiety (e.g., an oligophosphate comprising a thio-phosphate, a methylene, and/or other bridges between phosphates).

“Nucleotide analog,” as used herein refers to a chemical compound that is structurally similar to a nucleotide and capable of serving as a substrate or inhibitor of a nucleic acid polymerase. A nucleotide analog may have a modified or non-naturally occurring nucleobase moiety, a modified sugar, and/or a modified oligophosphate moiety.

“Nucleoside,” as used herein, refers to a molecular moiety that comprises a naturally occurring or non-naturally occurring nucleobase attached to a sugar moiety (e.g., ribose or deoxyribose).

“Nucleoside-5′-oligophosphate” or “N5OP,” as used herein, refers to a molecular moiety that comprises a ribose, deoxyribose, dideoxyribose (or derivatives thereof) having a naturally occurring or non-naturally occurring nucleobase attached to the 1′ position and an oligophosphate attached to the 5′ position. N5OPs include, but are not limited to, those have the following structure:

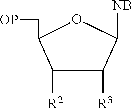

wherein NB is the nucleobase, OP is the oligophosphate, R² is selected from the group consisting of H and OH, and R³ is selected from the group consisting of H, OH, F, and —O—CH₃.

“Deoxynucleoside,” as used herein, refers to a molecular moiety that comprises a sugar moiety with a single hydroxyl group (e.g., deoxyribose or deoxyhexose group) to which is attached a naturally occurring or non-naturally occurring nucleobase.

“Oligophosphate,” as used herein, refers to a molecular moiety that comprises an oligomer of phosphate groups. For example, an oligophosphate can comprise an oligomer of from 2 to 20 phosphates, an oligomer of from 3 to 12 phosphates, an oligomer of from 3 to 9 phosphates.

“Polymerase,” as used herein, refers to any natural or non-naturally occurring enzyme or other catalyst that is capable of catalyzing a polymerization reaction, such as the polymerization of nucleotide monomers to form a nucleic acid polymer. Exemplary polymerases that may be used in the compositions and methods of the present disclosure include the nucleic acid polymerases such as DNA polymerase (e.g., enzyme of class EC 2.7.7.7), RNA polymerase (e.g., enzyme of class EC 2.7.7.6 or EC 2.7.7.48), reverse transcriptase (e.g., enzyme of class EC 2.7.7.49), and DNA ligase (e.g., enzyme of class EC 6.5.1.1).

“Nanopore,” as used herein, refers to a pore, channel, or passage formed or otherwise provided in a membrane or other barrier material that has a characteristic width or diameter of about 0.1 nm to about 1000 nm. A nanopore can be made of a naturally-occurring pore-forming protein, such as α-hemolysin from S. aureus, or a mutant or variant of a wild-type pore-forming protein, either non-naturally occurring (i.e., engineered) such as α-HL-C46, or naturally occurring. A membrane may be an organic membrane, such as a lipid bilayer, or a synthetic membrane made of a non-naturally occurring polymeric material. The nanopore may be disposed adjacent or in proximity to a sensor, a sensing circuit, or an electrode coupled to a sensing circuit, such as, for example, a complementary metal-oxide semiconductor (CMOS) or field effect transistor (FET) circuit.

“Pore-forming protein,” as used herein refers to a natural or non-naturally occurring protein capable of forming a pore or channel structure in a barrier material such as a lipid bilayer or cell membrane. The terms as used herein are intended to include both a pore-forming protein in solution, and a pore-forming protein embedded in a membrane or barrier material, or immobilized on a solid substrate or support. The terms as used herein are intended to including pore-forming proteins as monomers and also as any multimeric forms into which they are capable of assembling. Exemplary pore-forming proteins that may be used in the compositions and methods of the present disclosure include α-hemolysin (e.g., from S. aureus), β-hemolysin, γ-hemolysin, aerolysin, cytolysin (e.g., pneumolysis), leukocidin, melittin, and porin A (e.g., MspA from Mycobacterium smegmatis).

“Tag,” as used herein, refers to a molecule that enables or enhances the ability to detect and/or identify, either directly or indirectly, a molecule or molecular complex, which is coupled to the tag. For example, the tag can provide a detectable property or characteristic, such as steric bulk or volume, electrostatic charge, electrochemical potential, and/or spectroscopic signature.

“Tagged nucleotide,” as used herein refers to a nucleotide or nucleotide analog with a tag attached to the oligophosphate moiety, base moiety, or sugar moiety.

“Nanopore-detectable tag” as used herein refers to a tag that can enter into, become positioned in, be captured by, translocate through, and/or traverse a nanopore and thereby result in a detectable change in current through the nanopore. Exemplary nanopore-detectable tags include, but are not limited to, natural or synthetic polymers, such as polyethylene glycol, oligonucleotides, polypeptides, carbohydrates, peptide nucleic acid polymers, locked nucleic acid polymers, any of which may be optionally modified with or linked to chemical groups, such as dye moieties, or fluorophores, that can result in detectable nanopore current changes.

“Linker,” as used herein, refers to any molecular moiety that provides a bonding attachment with some space between two or more molecules, molecular groups, and/or molecular moieties.

“Peptide,” as used herein, refers to at least two amino acids covalently linked by an amide bond.

“Amino acid,” as used herein, refers to a compound comprising amine and carboxylic functional groups, and a side-chain. Amino acids can include the standard, 20 genetically encoded α-amino acids, as well as any other naturally-occurring and synthetic amino acids, known in the art and/or disclosed herein, which are capable of undergoing a condensation reaction with another amino acid to form a peptide.

“Polypeptide,” as used herein, refers to a polymer of from 2 to about 400 or more amino acids. When polypeptide sequences are presented herein as a string of one-letter or three-letter abbreviations (or mixtures thereof), the sequences are presented in the amino (N) to carboxy (C) direction in accordance with common convention.

“Helical structure,” as used herein, refers to an oligomer or polymer of amino acids that forms one or more three-dimensional spiral or loop structures, such as an α-helix structure.

“Overall charge,” as used herein in the context of polypeptide tags refers to the sum of the positively charged and negatively charged side-chains of the amino acid residues that make up the polypeptide tag. For example, a polypeptide tag comprising a polypeptide having 5 lysine residues, which are positively charged (+1), and 15 glutamic acid residues, which are negatively charged (−1), has an overall charge of −10.

“Background current” as used herein refers to the current level measured across a nanopore when a potential is applied and the nanopore is open and unblocked (e.g., there is no tag in the nanopore).

“Blocking current” as used herein refers to the current level measured across a nanopore when a potential is applied and a tag is present the nanopore. Generally, the presence of the tag molecule in the nanopore restrict the flow of charged molecules through the nanopore thereby altering the current level from the background.

“Blocking voltage” as used herein refers to the voltage level measured across a nanopore when a current is applied and a tag is present the nanopore. Generally, the presence of the tag molecule in the nanopore restrict the flow of charged molecules through the nanopore thereby altering the voltage level from the background

“Naturally occurring” refers to the form found in nature. For example, a naturally occurring or wild-type protein is a protein having a sequence present in an organism that can be isolated from a source found in nature, and which has not been intentionally modified by human manipulation.

“Non-naturally occurring” or “recombinant” or “engineered” or when used with reference to, e.g., nucleic acid, polypeptide, or a cell, refers to a material that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

### B. BASE-MODIFIED NUCLEOSIDE-5′-OLIGOPHOSPHATES

In an aspect, the present specification provides nucleoside-5′-oligophosphates (N5OP) comprising a nucleobase bearing a positively charged moiety (PCM), also referred to as a base-modified N5OP (bm-N5OP). Naturally occurring nucleic acids generally have a large net-negative charge, owing to presence of multiple phosphodiester bonds linking adjoining nucleotides. In contrast, when the presently disclosed nucleotides are incorporated into nucleic acids, the PCM neutralizes at least a portion of the negative charge, thereby reducing the overall net charge of the nucleic acid compared to a nucleic acid having the same sequence of naturally occurring nucleotides.

In an embodiment, the nucleobases comprising PCM is included in a N5OP according to the structure of Formula 1:

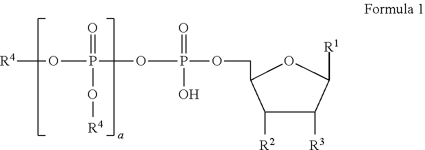

wherein R¹ is the nucleobase comprising PCM, R² is selected from the group consisting of H and OH; R³ is selected from the group consisting of H, OH, F, and —O—CH₃; R⁴ is H or a nanopore-detectable tag construct; and a is from 2 to 12. In an embodiment, R² is OH and R³ is H. In another embodiment, R² is OH and R³ is OH. In another embodiment, R² is H and R³ is H.

Any positively charged group that is compatible with polymerase-based nucleic acid amplification may be used to confer the net-positive charge on PCM, including but not limited to primary amines, secondary amines, tertiary amines (including cyclic amines), quaternary amines, guanidinium groups, and heteroaromatic rings. In an embodiment, PCM has a structure according to Formula 2:

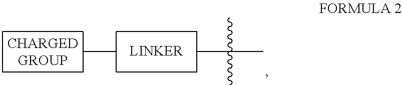

wherein CHARGED GROUP is the positively charged group and LINKER is a linker used to covalently linked the CHARGED GROUP to the nucleobase. It is contemplated that a wide range of linkers can be used to covalently couple the charged group to the nucleobase. Generally, the linker can comprise any molecular moiety that is capable of providing a covalent coupling and a spacing or structure between the compound and the charged moiety. Such linker parameters can be routinely determined by the ordinary artisan using methods known in the art. In an exemplary embodiment, LINKER is selected from the group consisting of an alkane, an alkene, an alkyne, an aryl group, a heteroaryl group, an amide, an ether, and a polyether. Exemplary PCM structures within the scope of Formula 2 include:

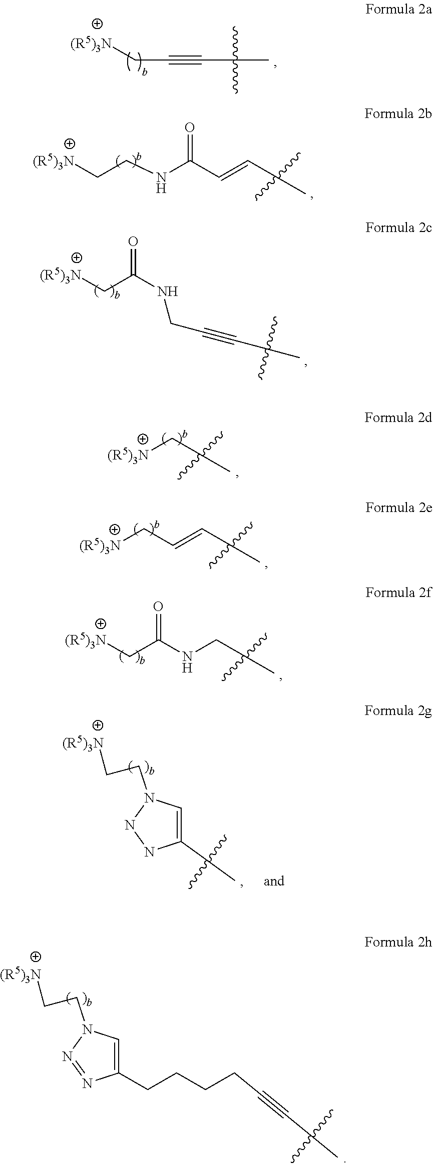

wherein R⁵ is selected from the group consisting of H, F, Cl, Br, alkyl, alkyl halide, alkyl ether, alkyl amine, and b is from 1 to 12 (including, for example, from 1 to 8, from 1 to 6, from 1 to 4). In an embodiment, R⁵ is H.

Any method of adding positively charged moieties to nucleobases may be used, so long as the resulting N5OP (a) is capable of being polymerized into a nucleic acid in a template-dependent manner and (b) possesses the ability to base pair with a naturally occurring nucleotide. For example, R1 may be a 7-deazapurine derivative,
such as

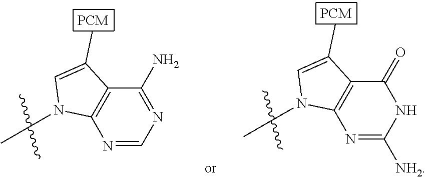 [SMILES: *n1cc(C)c2c(N)ncnc21 ; *n1cc(C)c2c(=O)[nH]c(N)nc21]

Exemplary methods of adding moieties to the 7 position of 7-deazapurines include using standard transition metal catalyzed cross coupling reaction of a 7-halo-deazaG or 7-halo-deazaA with the appropriate substrate (amine, alkyne, alkene, etc), such as Suzuki, Sonogashira, or Heck coupling reactions. As another example, R¹ may be an 8-substituted purine, such as

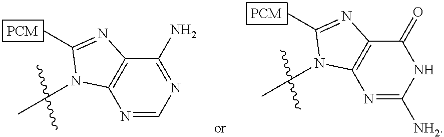 [SMILES: *n1c(C)nc2c(N)ncnc21 ; *n1c(C)nc2c(=O)[nH]c(N)nc21]

Exemplary methods of generating 8-substituted purines include using standard transition metal catalyzed cross coupling reaction of a 8-halo-purine with the appropriate substrate (amine, alkyne, alkene, etc.), such as Suzuki, Sonogashira, or Heck coupling reactions. In another example, the R1 may be an adenosine derivative having PCM attached to the amine group at the 6 position, such as a nucleobase according to the following structure:

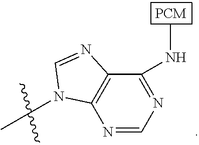 [SMILES: *n1cnc2c(NC)ncnc21]

Exemplary methods of making such modifications to adenosine include coupling or substitution reactions of 6-chloropurine with an amine, such as by the reaction scheme outlined by Liu. As another example, R1 may be a guanosine derivative having PCM attached to the amine group at the 2 position, such as a nucleobase according to the following structure:

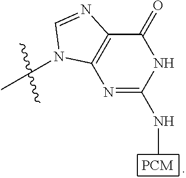 [SMILES: *n1cnc2c(=O)[nH]c(NC)nc21]

Exemplary methods of making such modifications to guanosine include using standard transition metal catalyzed cross coupling reaction of a 2-halo-dG derivative with an amine, such as Suzuki, Sonogashira, or Heck coupling reactions. As another example, R1 may be a 5-substitute pyrimidine, such as nucleobases having structures according to

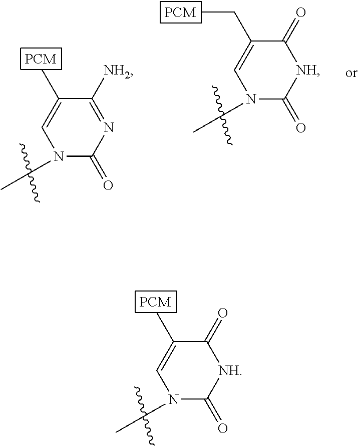

Exemplary methods of making 5-substituted pyrimidines include using standard transition metal catalyzed cross coupling reaction of a 5-halo-dT with the appropriate substrate (amine, alkyne, alkene, etc), such as Suzuki, Sonogashira, or Heck coupling reactions. As yet another example, R¹ may be a cytosine derivative having PCM attached to the amine group at the 4-position, such as the following structure

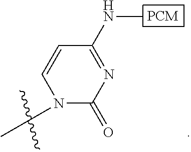 [SMILES: *n1ccc(NC)nc1=O]

Exemplary methods of making 4-substituted cytosines include using a dC intermediate as described by Cismas & Gimisis or a “convertible dC” nucleotide treated with an amine derivative.

In an embodiment, R4 is H (i.e., the oligophosphate of the bm-N5OP does not comprise a nanopore-detectable tag). In the context of tag-based SBS, such embodiments may be especially useful for generating template nucleic acids and/or primers for use on an SBS system.

In another embodiment, one instance of R4 is the nanopore-detectable tag, and the remaining instances of R4 are H (i.e., the oligophosphate of the bm-N5OP comprises a single nanopore-detectable tag). In an embodiment, the nanopore-detectable tag is tag that affects a charge characteristic of the nanopore, such as polyethylene glycol (PEG) tags, nucleotide containing tags, polypeptide-containing tags, or other charged polymers, including, for example, those disclosed by U.S. Pat. Nos. 8,652,779, 10,246,479, 10,443,096, WO 2017-042038, WO 2018-037096, WO 2018-191389, and WO 2019-166457 (each of which is incorporated herein by reference). In an embodiment, the nanopore-detectable tag has a net-negative charge.

### C. ISOLATED NUCLEIC ACIDS INCLUDING NUCLEOBASES COMPRISING A PCM AND METHODS OF MAKING THE SAME

Also disclosed herein are nucleic acids comprising at least one nucleobase having a positively charged moiety (PCM) as disclosed herein. As used herein, the nucleobase comprising the PCM shall be referred to as a base-modified nucleobase. In some embodiments, at least 5% of the nucleobases of the nucleic acid are base-modified nucleobases. In some embodiments, at least 10% of the nucleobases of the nucleic acid are base-modified nucleobases. In some embodiments, at least 15% of the nucleobases of the nucleic acid are base-modified nucleobases. In some embodiments, at least 20% of the nucleobases of the nucleic acid are base-modified nucleobases. In some embodiments, at least 25% of the nucleobases of the nucleic acid are base-modified nucleobases. In some embodiments, at least 30% of the nucleobases of the nucleic acid are base-modified nucleobases. In some embodiments, at least 35% of the nucleobases of the nucleic acid are base-modified nucleobases. In some embodiments, at least 40% of the nucleobases of the nucleic acid are base-modified nucleobases. In some embodiments, at least 45% of the nucleobases of the nucleic acid are base-modified nucleobases. In some embodiments, at least 50% of the nucleobases of the nucleic acid are base-modified nucleobases. In some embodiments, at least 55% of the nucleobases of the nucleic acid are base-modified nucleobases. In some embodiments, at least 60% of the nucleobases of the nucleic acid are base-modified nucleobases. In some embodiments, at least 65% of the nucleobases of the nucleic acid are base-modified nucleobases. In some embodiments, at least 70% of the nucleobases of the nucleic acid are base-modified nucleobases. In some embodiments, at least 75% of the nucleobases of the nucleic acid are base-modified nucleobases. In some embodiments, at least 80% of the nucleobases of the nucleic acid are base-modified nucleobases. In some embodiments, at least 85% of the nucleobases of the nucleic acid are base-modified nucleobases. In some embodiments, at least 90% of the nucleobases of the nucleic acid are base-modified nucleobases. In some embodiments, at least 95% of the nucleobases of the nucleic acid are base-modified nucleobases.

Exemplary nucleobase structures useful in the nucleic acids include:

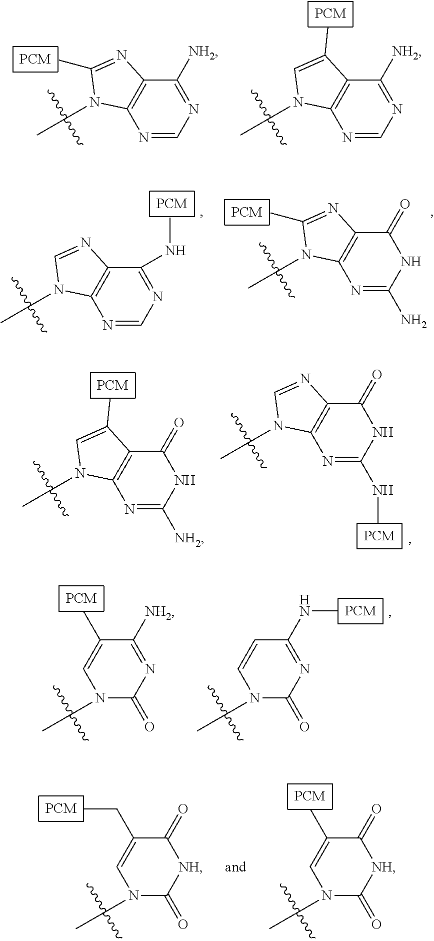

In an embodiment, PCM of the base-modified nucleobase has a structure according to Formula 2:

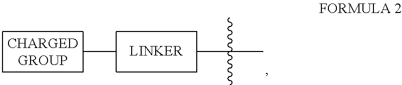

wherein CHARGED GROUP is the positively charged group and LINKER is a linker used to covalently linked the CHARGED GROUP to the nucleobase. It is contemplated that a wide range of linkers can be used to covalently couple the charged group to the nucleobase. Generally, the linker can comprise any molecular moiety that is capable of providing a covalent coupling and a spacing or structure between the nucleobase and the charged moiety. In an exemplary embodiment, LINKER is selected from the group consisting of an alkane, an alkene, an alkyne, an aryl group, a heteroaryl group, an amide, an ether, and a polyether. Exemplary PCM structures within the scope of Formula 2 include:

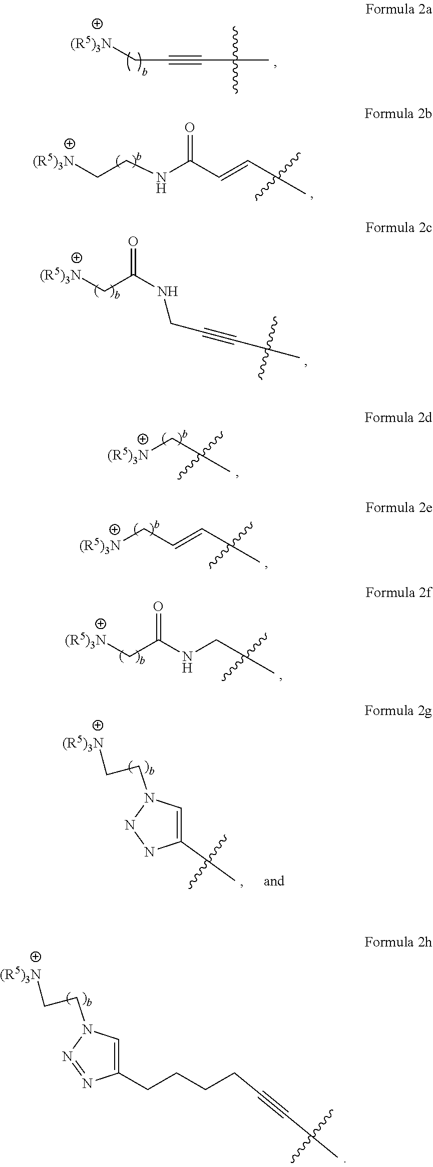

wherein R⁵ is selected from the group consisting of H, F, Cl, Br, alkyl, alkyl halide, alkyl ether, alkyl amine, and b is from 1 to 12 (including, for example, from 1 to 8, from 1 to 6, from 1 to 4). In an embodiment, R⁵ is H.

Such nucleic acids may be useful, for example, as a template nucleic acid and/or as a primer nucleic acid for performing tag-base SBS reactions. Because many nanopores bear a net-positive charge, the high concentration of negative charge on the template nucleic acid and primer may cause those entities to be attracted into the channel of the nanopore. Repeated insertions may show up as a persistent background band in sequencing runs, while threading of the template through the nanopore may render the nanopore inactive. To mitigate this effect, nucleobases having a PCM attached thereto are added into the template nucleic acid. Without being bound by theory, the positive charge of the PCM neutralizes at least a portion of the net-negative charge of the template nucleic acid or primer, thereby reducing the attraction between positively charged nanopore and the nucleic acid. The amount of nucleobase including the PCM that is incorporated into the template and/or primer can be selected such that a sequencing run with reduced background is observed relative to a template and/or primer containing only native nucleobases.

Any method of generating a nucleic acid with native nucleotides may also be used to generate the presently described nucleic acids. For example, a polymerase chain reaction (PCR) may be conducted to polymerize a set of N5OPs comprising one or more of the bm-N5OPs described herein. By varying the ratio of native nucleotides to bm-N5OPs in the PCR process, the percentage of nucleobases having the PCM can be altered to obtain the desired degree of neutralizing effect on the net charge.

### D. SETS OF N5OP AND KITS CONTAINING THE SAME

Also disclosed herein are sets of N5OPs. As used herein a “set” of N5OPs is a grouping of N5OP that are useful together for a specific application, such as for generating a template nucleic acid, a primer nucleic acid, or for performing tag-based sequencing-by-synthesis.

In an embodiment, a set of N5OP is provided comprising, consisting essentially of, or consisting of:
an adenosine-5′-oliogophosphate (A5OP);
a cytidine-5′-oliogophosphate (C5OP);
a guanosine-5′-oliogophosphate (G5OP); and
a thymidine-5′-oliogophosphate (T5OP) and/or a uridine-5′-oliogophosphate (U5OP);
   wherein at least 1 of A5OP, C5OP, G5OP, and T5OP and/or U5OP is a bm-N5OP as described herein. In some cases, at least 2 of A5OP, C5OP, G5OP, and T5OP and/or U5OP are bm-N5OP as described herein. In some cases, at least 3 of A5OP, C5OP, G5OP, and T5OP and/or U5OP are bm-N5OP as described herein. In some cases, each of A5OP, C5OP, G5OP, and T5OP and/or U5OP is a bm-N5OP as described herein.

In some cases, the set further comprises one or more N5OPs that (a) is not base modified and (b) has a base corresponding to one of the bm-N5OP(s) of the set. For example, the set of dN5OP may include both an A5OP and a bmA5OP. Such embodiments might be desirable, for example, where it is desired to control the amount of bm-N5OP that is included in the template. For example, where it is desired to have a template nucleic acid in which not more than 25% of G5OPs are base modified, the set may include both a G5OP and a bmG5OP.

In some cases, the set may include one or more dideoxynucleoside-5-oligophosphates (ddN5OP). ddN5OPs can be incorporated into a nucleic acid by a PCR reaction, but further polymerization cannot occur because no hydroxyl group is at the 3′ position. ddN5OPs are used in many sequencing methods, including Sanger sequencing. In the context of tag-based SBS, ddN5OPs could be used to increase the certainty of the base immediately following the ddN5OP incorporated into a growing amplicon. Because the base immediately following the ddN5OP can still occupy the polymerase, but will not be polymerized into the amplicon, the amount of time it occupies the polymerase will be significantly increased relative to other nucleotides. This would enable hundreds of captures of the associated tag, which substantially increases the confidence in the identity of the nucleotide following the ddN5OP. This may be especially useful for short reads where a high degree of confidence is needed at each position of the template, for example, for detection of single nucleotide polymorphisms.

In an embodiment, the A5OP, the C5OP, the G5OP, and the T5OP and/or U5OP are deoxyribonucleotides (dA5OP, dC5OP, dG5OP, dT/dU5OP, and dU5OP, respectively). For example, when the set of N5OPs is intended to be used to generate a DNA template or a DNA-based primer, the set of N5OPs may comprise a dA5OP, a dC5OP, dG5OP, and dT/dU5OP, with the proviso that at least one of the dA5OP, dC5OP, dG5OP, and dT/dU5OP is a base-modified deoxyribonucleoside-5′-oligophosphate (bm-dN5OP). Exemplary sets that include bm-dN5OPs are set forth in Table 1:

TABLE 1

| SET | | |
|---|---|---|
| # | Base-Modified dN5OPs | Non-Base-Modified dN5OPs |
| 1 | bm-dA5OP | dC5OP, dT/dU5OP, and dG5OP |
| 2 | bm-dC5OP | dA5OP, dT/dU5OP, and dG5OP |
| 3 | bm-dT/dU5OP | dA5OP, dC5OP, and dG5OP |
| 4 | bm-dG5OP | dA5OP, dC5OP, and dT/dU5OP |
| 5 | bm-dA5OP, bm-dC5OP | dT/dU5OP and dG5OP |
| 6 | bm-dA5OP, bm-dC5OP, and | dG5OP |
| | bm-dT/dU5OP | |
| 7 | bm-dA5OP, bm-dC5OP, | None |
| | bm-dT/dU5OP, and bm-dG5OP | |
| 8 | bm-dA5OP and bm-dT/dU5OP | dC5OP and dG5OP |
| 9 | bm-dA5OP, bm-dT/dU5OP, and | dC5OP |
| | bm-dG5OP | |
| 10 | bm-dA5OP and bm-dG5OP | dC5OP and dT/dU5OP |
| 11 | bm-dC5OP and bm-dT/dU5OP | dA5OP and dG5OP |
| 12 | bm-dC5OP, bm-dT/dU5OP, and | dA5OP |
| | bm-dG5OP | |
| 13 | bm-dC5OP and bm-dG5OP | dA5OP and dT/dU5OP |
| 14 | bm-dT/dU5OP and bm-dG5OP | dA5OP and dC5OP |
| 15 | bm-dA5OP | dA5OP, dC5OP, dT/dU5OP, |
| | | dG5OP |
| 16 | bm-dC5OP | dA5OP, dC5OP, dT/dU5OP, |
| | | dG5OP |
| 17 | bm-dT/dU5OP | dA5OP, dC5OP, dT/dU5OP, |
| | | dG5OP |
| 18 | bm-dG5OP | dA5OP, dC5OP, dT/dU5OP, |
| | | dG5OP |
| 19 | bm-dA5OP and bm-dC5OP | dA5OP, dC5OP, dT/dU5OP, |
| | | dG5OP |
| 20 | bm-dA5OP and bm-dT/dU5OP | dA5OP, dC5OP, dT/dU5OP, |
| | | dG5OP |
| 21 | bm-dA5OP and bm-dG5OP | dA5OP, dC5OP, dT/dU5OP, |
| | | dG5OP |
| 22 | bm-dC5OP and bm-dT/dU5OP | dA5OP, dC5OP, dT/dU5OP, |
| | | dG5OP |
| 23 | bm-dC5OP and bm-dG5OP | dA5OP, dC5OP, dT/dU5OP, |
| | | dG5OP |
| 24 | bm-dT/dU5OP and bm-dG5OP | dA5OP, dC5OP, dT/dU5OP, |
| | | dG5OP |
| 25 | bm-dA5OP, bm-dC5OP, and | dA5OP, dC5OP, dT/dU5OP, |
| | bm-dT/dU5OP | dG5OP |
| 26 | bm-dA5OP, bm-dC5OP, and | dA5OP, dC5OP, dT/dU5OP, |
| | bm-dG5OP | dG5OP |
| 27 | bm-dA5OP, bm-dT/dU5OP, and | dA5OP, dC5OP, dT/dU5OP, |
| | bm-dG5OP | dG5OP |
| 28 | bm-dC5OP, bm-dT/dU5OP, and | dA5OP, dC5OP, dT/dU5OP, |
| | bm-dG5OP | dG5OP |
| 29 | bm-dC5OP, bm-dC5OP, | dA5OP, dC5OP, dT/dU5OP, |
| | bm-dT/dU5OP, and bm-dG5OP | dG5OP |

Table 1 is not intended to be an exhaustive list of all sets of dN5OPs described by this paragraph.

In another embodiment, the A5OP, the C5OP, the G5OP, and the T5OP and/or U5OP are ribonucleotides (rA5OP, rC5OP, rG5OP, rT5OP, and rU5OP, respectively). For example, when the set of N5OPs is intended to be used to generate an RNA template or an RNA-based primer, the set of N5OPs may comprise a rA5OP, a rC5OP, rG5OP, and rT/rU5OP, with the proviso that at least one of the rA5OP, dC5OP, dG5OP, and rT/rU5OP is a base-modified ribonucleoside-5′-oligophosphate (bm-rN5OP). Exemplary sets that include bm-rN5OPs are set forth in Table 2:

TABLE 2

| SET | | |
|---|---|---|
| # | Base-Modified rN5OPs | Non-Base-Modified rN5OPs |
| 1 | bm-rA5OP | rC5OP, rT/rU5OP, and rG5OP |
| 2 | bm-rC5OP | rA5OP, rT/rU5OP, and rG5OP |
| 3 | bm-rT/rU5OP | rA5OP, rC5OP, and rG5OP |
| 4 | bm-rG5OP | rA5OP, rC5OP, and rT/rU5OP |
| 5 | bm-rA5OP, bm-rC5OP | rT/rU5OP and rG5OP |
| 6 | bm-rA5OP, bm-rC5OP, and | rG5OP |
| | bm-rT/rU5OP | |
| 7 | bm-rA5OP, bm-rC5OP, | None |
| | bm-rT/rU5OP, and bm-rG5OP | |
| 8 | bm-rA5OP and bm-rT/rU5OP | rC5OP and rG5OP |
| 9 | bm-rA5OP, bm-rT/rU5OP, and | rC5OP |
| | bm-rG5OP | |
| 10 | bm-rA5OP and bm-rG5OP | rC5OP and rT/rU5OP |
| 11 | bm-rC5OP and bm-rT/rU5OP | rA5OP and rG5OP |
| 12 | bm-rC5OP, bm-rT/rU5OP, and | rA5OP |
| | bm-rG5OP | |
| 13 | bm-rC5OP and bm-rG5OP | rA5OP and rT/rU5OP |
| 14 | bm-rT/rU5OP and bm-rG5OP | rA5OP and rC5OP |
| 15 | bm-rA5OP | rA5OP, rC5OP, rT/rU5OP, |
| | | rG5OP |
| 16 | bm-rC5OP | rA5OP, rC5OP, rT/rU5OP, |
| | | rG5OP |
| 17 | bm-rT/rU5OP | rA5OP, rC5OP, rT/rU5OP, |
| | | rG5OP |
| 18 | bm-rG5OP | rA5OP, rC5OP, rT/rU5OP, |
| | | rG5OP |
| 19 | bm-rA5OP and bm-rC5OP | rA5OP, rC5OP, rT/rU5OP, |
| | | rG5OP |
| 20 | bm-rA5OP and bm-rT/rU5OP | rA5OP, rC5OP, rT/rU5OP, |
| | | rG5OP |
| 21 | bm-rA5OP and bm-rG5OP | rA5OP, rC5OP, rT/rU5OP, |
| | | rG5OP |
| 22 | bm-rC5OP and bm-rT/rU5OP | rA5OP, rC5OP, rT/rU5OP, |
| | | rG5OP |
| 23 | bm-rC5OP and bm-rG5OP | rA5OP, rC5OP, rT/rU5OP, |
| | | rG5OP |
| 24 | bm-rT/rU5OP and bm-rG5OP | rA5OP, rC5OP, rT/rU5OP, |
| | | rG5OP |
| 25 | bm-rA5OP, bm-rC5OP, and | rA5OP, rC5OP, rT/rU5OP, |
| | bm-rT/rU5OP | rG5OP |
| 26 | bm-rA5OP, bm-rC5OP, and | rA5OP, rC5OP, rT/rU5OP, |
| | bm-rG5OP | rG5OP |
| 27 | bm-rA5OP, bm-rT/rU5OP, and | rA5OP, rC5OP, rT/rU5OP, |
| | bm-rG5OP | rG5OP |
| 28 | bm-rC5OP, bm-rT/rU5OP, and | rA5OP, rC5OP, rT/rU5OP, |
| | bm-rG5OP | rG5OP |
| 29 | bm-rC5OP, bm-rC5OP, | rA5OP, rC5OP, rT/rU5OP, |
| | bm-rT/rU5OP, and bm-rG5OP | rG5OP |

Table 2 is not intended to be an exhaustive list of all sets of rN5OPs described by this paragraph.

In an embodiment, some or all of the N5OPs of the set may comprise a tag. As one example, N5OPs comprising a tag may be used to generate a template or primer nucleic acid. When the tag is located on one of the phosphate groups, the tag is released upon incorporation into the template or the primer and therefore will not present an issue during a tag-based SBS run. As another example, where the set of N5OPs is intended to be used on a nanopore-based sequencing system for sequencing a template nucleic acid in a tag-based-SBS method, each of the N5OPs should be tagged. In this context, the tags are selected such that the base with which it is associated is distinguishable from the other bases of the set. In this context, the bm-N5OP preferably has the same tag as its corresponding non-base modified N5OP, if both are included in the set. In an exemplary embodiment, a set of dN5OPs according to Table 2 is provided, in which each dN5OP and bm-dN5OP is tagged. In an exemplary embodiment, a set of rN5OPs according to Table 2 is provided, in which each rN5OP and bm-rN5OP is tagged.

In some embodiments, the sets of N5OP are provided in a kit, for example. In one embodiment, the N5OPs may be present in the kit in a solid form (such as salts, crystals, lyophilates, or the like), which kit may optionally include a diluent for dissolving the solid for use and for diluting the N5OPs to a final useful concentration. In another embodiment, the N5OPs may be present in the kit in a concentrate format, which kit may optionally include a diluent for diluting the N5OPs to a final concentration. As used herein, a “concentrate format” is a format in which the N5OPs are provided in solution at a higher concentration than the concentration at which they are intended to be input into a system (such as a PCR system or a nanopore-based sequencing system). In another embodiment, the N5OPs may be present in the kit in a ready-to-use form. As used herein, a “ready-to-use” format is a format in which the N5OPs are provided in solution at the final concentration at which they are intended to be implemented on a system (such as a PCR system or a nanopore sequencing system). In another embodiment, the concentrate format or ready-to-use format is provided as a “master mix” that includes at least the set of N5OPs, a polymerase, and one or more ancillary reagents necessary for the polymerase to catalyze a template-dependent polymerase chain reaction with the N5OPs. The N5OPs may be present in the kit separately, or may be pre-mixed with one another in a pre-determined ratio.

The kits may be useful, for example, for generating a template nucleic acid to be used on a tag-based SBS system. In such an embodiment, the kit may further comprise, for example, a polymerase useful for transforming a target nucleic acid to a template nucleic acid, as well as ancillary reagents for performing a polymerase chain reaction to generate the template nucleic acid from the target nucleic acid, such as buffers, cofactors, catalyzers, primers, and the like. The N5OPs may or may not be tagged.

As another example, the kits may be useful for sequencing a template nucleic acid on a tag-based SBS system. In such an example, the kit may further comprise, for example, a polymerase useful for generating an amplicon of the template nucleic acid, a nanopore or peptides useful for generating a nanopore, as well as ancillary reagents for performing a polymerase chain reaction, such as buffers, cofactors, catalyzers, primers, and the like. In such an example, the N5OPs are tagged. In this context, the tags are selected such that they generate a unique electronic signature when occupying the nanopore, which allows the nucleobase with which the tag is associated to be distinguishable from the other nucleobases of the set. In this context, the bm-N5OP preferably has the same tag as its corresponding non-base modified N5OP, if both are included in the set. Exemplary tags include, for example, tags based on polypeptides, polynucleotides, and polyethylene glycol. See, e.g., U.S. Pat. No. 8,652,779 and WO2017042038A1.

Exemplary polymerases useful in the present kits include those derived from DNA polymerase Clostridium phage phiCPV4 (described by GenBank Accession No. YP_00648862, referred to herein as “Pol6”), phi29 DNA polymerase, T7 DNA pol, T4 DNA pol, E. coli DNA pol 1, Klenow fragment, T7 RNA polymerase, and E. coli RNA polymerase, as well as associated subunits and cofactors. In an embodiment, the polymerase is a DNA polymerase derived from Pol6. Exemplary Pol6 derivatives useful in nanopore-based sequencing are disclosed at, for example, US 2016/0222363, US 2016/0333327, US 2017/0267983, US 2018/0094249, and US 2018/0245147.

Exemplary nanopore-forming proteins useful in the present kits include those based on α-hemolysin (αHL), outer membrane porin G (OmpG), Mycobacterium smegmatis porin A (MspA), leukocidin nanopore, outer membrane porin F (OmpF) nanopore, cytolysin A (ClyA) nanopore, outer membrane phospholipase A nanopore, Neisseria autotransporter lipoprotein (NalP) nanopore, WZA nanopore, Nocardia farcinica NfpA/NfpB cationic selective channel nanopore, lysenin nanopore, aerolysin, and Curlin sigma S-dependent growth subunit G (CsgG) nanopore. In some embodiments, the nanopore-forming protein is based on αHL, wherein the kit comprises a preparation of a polypeptide comprising an amino acid sequence having at least 75%, at least 80%, at least 85%, at least 90%, or at least 95% sequence identity to SEQ ID NO: 1. In some embodiments, the kit comprises a preparation of a polypeptide comprising the amino acid sequence having at least 75%, at least 80%, at least 85%, at least 90%, or at least 95% sequence identity to SEQ ID NO: 1, wherein a portion of the polypeptides in the preparation is bound to or adapted to be bound to a polymerase. Exemplary methods of attaching a polymerase to an αHL nanopore include SpyTag/SpyCatcher peptide system (Zakeri et al. PNAS 109: E690-E697 2012), native chemical ligation system (Thapa et al., Molecules 19:14461-14483 2014), sortase system (Wu and Guo, J Carbohydr Chem 31:48-66 2012; Heck et al., Appl Microbiol Biotechnol 97:461-475 2013)), transglutaminase systems (Dennler et al., Bioconjug Chem 25:569 578 2014), formylglycine linkage systems (Rashidian et al., Bio conjug Chem 24:1277-1294 2013), Click chemistry attachment systems, or other chemical ligation techniques known in the art. In another embodiment, the kit comprises a preparation of a polypeptide comprising the amino acid sequence having at least 75%, at least 80%, at least 85%, at least 90%, or at least 95% sequence identity to SEQ ID NO: 1, wherein a portion of the polypeptides in the preparation are fusion proteins with the polymerase. In another embodiment, the kit comprises a preparation of a first polypeptide and a preparation of a second polypeptide, each of the first and second polypeptides comprising an amino acid sequence having at least 75%, at least 80%, at least 85%, at least 90%, or at least 95% sequence identity to SEQ ID NO: 1, wherein the first polypeptide is bound to or adapted to be bound to a polymerase, and the second polypeptide is not bound to or adapted to be bound to a polymerase.

### E. NUCLEIC ACID SEQUENCING SYSTEMS AND METHODS

Systems and methods for performing nucleic acid sequencing using the disclosed bm-N5OPs disclosed herein and/or nucleic acids comprising the base-modified nucleobases are also included.

Systems for nanopore-based nucleic acid sequencing generally comprise a chip with a plurality of nanopore sequencing complexes and a computing system adapted to record changes in one or more electrical characteristics of the nanopore sequencing complexes.

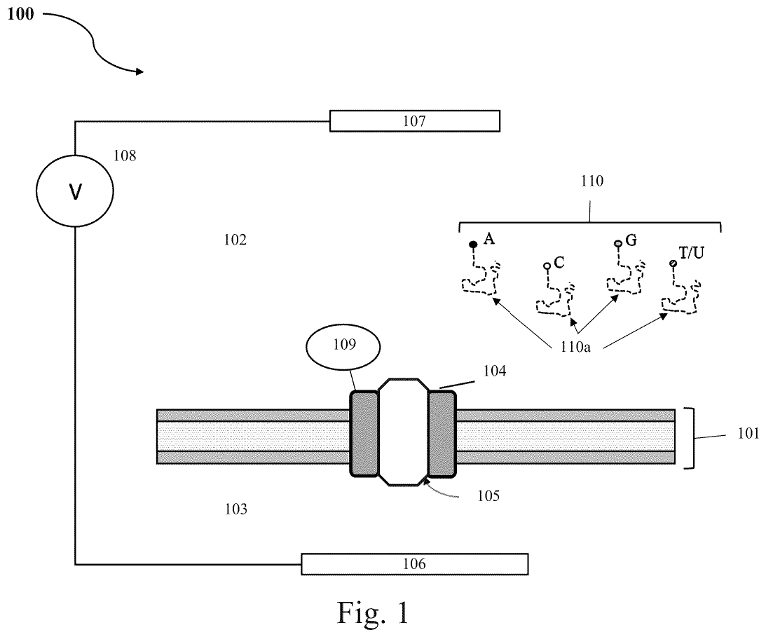

FIG. 1 illustrates an exemplary nanopore sequencing complex 100. An electrochemically resistive barrier 101 separates a first electrolyte solution 102 from a second electrolyte solution 103. The side of the barrier on which the first electrolyte solution is disposed is termed the cis side of the barrier, which the side on which the second electrolyte solution is disposed is termed the trans side. A nanopore 104 is inserted into the barrier 101, such that the channel 105 permits ion exchange between the first electrolyte solution and the second electrolyte solution. In the context of the present systems, the channel 105 has a net-positive charge. As used in this context, the net charge of channel 105 is determined by summing the net charge of the side chains of all of the solvent facing residues in the channel at pH 7.0. A working electrode 106 and a counter electrode 107 are operatively coupled to a signal source 108. The signal source 108 applies a voltage signal between the working electrode 106 and the counter electrode 107. The nanopore 104 is positioned with respect to the electrodes such that changes in at least one electrical characteristic of the nanopore can be detected and transmitted to the computing system. Where the system is used for sequencing-by-synthesis methods, the system further comprises a nucleic acid polymerase 109 associated with the nanopore on the cis side of the barrier; and a set of polymer-tagged N5OP 110 disposed in the first electrolyte solution. Each nucleotide of the set comprises a tag 110a. In one embodiment, the set of N5OP comprise one of more bm-N5OP as disclosed herein (such as the sets of N5OP disclosed herein). In an alternative embodiment, the set of N5OP does not comprise any base-modified N5OP as disclosed herein.

Any semi-permeable membrane that permits the transmembrane flow of water but has limited to no permeability to the flow of ions or other osmolytes may be used as an electrochemically-resistive barrier, so long as the nanopore can be inserted. For example, the disclosed methods and systems can be used with membranes that are polymeric. In some embodiments, the membrane is a copolymer. In some embodiments, the membrane is a triblock copolymer. In an exemplary embodiment, the membrane is an A-B-A triblock copolymer wherein “A” is poly-b-(methyloxazoline) and “B” is poly(dimethylsiloxane)-poly-b-(methyloxazoline) (Pmoxa-PDMS-Pmoxa membrane). In other embodiments, the electrochemically-resistive barrier may be a lipid bilayer. Exemplary materials used to form lipid bilayers include, for example, phospholipids, for example, selected from diphytanoyl-phosphatidylcholine (DPhPC), 1,2-diphytanoyl-sn-glycero-3-phosphocholine, 1,2-di-O-phytanyl-sn-glycero-3-phosphocholine (DOPhPC), palmitoyl-oleoyl-phosphatidylcholine (POPC), dioleoyl-phosphatidyl-methylester (DOPME), dipalmitoylphosphatidylcholine (DPPC), phosphatidylcholine, phosphatidylethanolamine, phosphatidylserine, phosphatidic acid, phosphatidylinositol, phosphatidylglycerol, sphingomyelin, 1,2-di-O-phytanyl-sn-glycerol, 1,2-dipalmitoyl-sn-glycero-3-phosphoethanolamine-N-[methoxy(polyethylene glycol)-350], 1,2-dipalmitoyl-sn-glycero-3-phosphoethanolamine-N-[methoxy(polyethylene glycol)-550], 1,2-dipalmitoyl-sn-glycero-3-phosphoethanolamine-N-[methoxy(polyethylene glycol)-750], 1,2-dipalmitoyl-sn-glycero-3-phosphoethanolamine-N-[methoxy(polyethylene glycol)-1000], 1,2-dipalmitoyl-sn-glycero-3-phosphoethanolamine-N-[methoxy(polyethylene glycol)-7000], 1,2-dioleoyl-sn-glycero-3-phosphoethanolamine-N-lactosyl, GM1 Ganglioside, Lysophosphatidylcholine (LPC), or any combination thereof.

The electrochemically-resistive barrier 101 separates the second electrolyte solution 103 on the trans side of the barrier from the first electrolyte solution 102 on the cis side of the barrier. The first electrolyte 102 and second electrolyte 103 are aqueous solutions buffered to an optimum ion concentration and maintained at an optimum pH to keep the nanopore open and the barrier intact as long as possible. The first electrolyte solution can comprise free nanopores (prior to insertion in the barrier), a template nucleic acid, and any ancillary reagents needed to sequence the nucleic acid of interest (such as primer nucleic acids and the set of N5OPs for SBS sequencing methods). The first and second electrolyte solutions may further comprise one or more of the following: lithium chloride (LiCl), sodium chloride (NaCl), potassium chloride (KCl), lithium glutamate, sodium glutamate, potassium glutamate, lithium acetate, sodium acetate, potassium acetate, calcium chloride (CaCl2), strontium chloride (SrCl2), manganese chloride (MnCl2), and magnesium chloride (MgCl2). In one embodiment, at least the primer nucleic acid comprises one or more of the base-modified nucleobases disclosed herein. In another embodiment, at least the template nucleic acid comprises one or more of the base-modified nucleobases disclosed herein. In another embodiment, both the primer nucleic acid and the template nucleic acid comprise one or more of the base-modified nucleobases disclosed herein. In another embodiment, the set of N5OPs comprises one or more one or more of the bm-N5OP disclosed herein. In another embodiment, the primer nucleic acid comprises one or more of the base-modified nucleobases disclosed herein and the set of N5OPs comprises one or more bm-N5OPs as disclosed herein. In yet another embodiment, the template nucleic acid comprises one or more of the base-modified nucleobases disclosed herein and the set of N5OPs comprises one or more bm-N5OPs as disclosed herein. In yet another embodiment, the primer nucleic acid comprises one or more of the base-modified nucleobases disclosed herein, the template nucleic acid comprises one or more of the base-modified nucleobases disclosed herein, and the set of N5OPs comprises one or more bm-N5OPs as disclosed herein.

A single free nanopore (not illustrated) can be inserted into barrier 101 by an electroporation process caused by the voltage signal, thereby forming a nanopore 104 in barrier 101. The channel 105 crosses the barrier 101 and provides the only path for ionic flow from the first electrolyte 102 to working electrode 106.

In some embodiments, working electrode 106 is a metal electrode. For non-faradaic conduction, working electrode 106 can be made of metals or other materials that are resistant to corrosion and oxidation, such as, for example, platinum, gold, titanium nitride, and graphite. For example, working electrode 106 can be a platinum electrode with electroplated platinum. In another example, working electrode 106 can be a titanium nitride (TiN) working electrode. Working electrode 106 can be porous, thereby increasing its surface area and a resulting capacitance associated with working electrode 106. Because the working electrode of a nanopore sequencing complex can be independent from the working electrode of another nanopore sequencing complex, the working electrode can be referred to as cell electrode in this disclosure.

Counter electrode (CE) 107 can be an electrochemical potential sensor. In some embodiments, counter electrode 107 is shared between a plurality of nanopore sequencing complexes, and can therefore be referred to as a common electrode. The common electrode can be configured to apply a common potential to the first electrolyte 102 in contact with the nanopore 104. Counter electrode 107 and working electrode 106 can be coupled to signal source 108 for providing electrical stimulus (e.g., voltage bias) across barrier 101, and can be used for sensing electrical characteristics of barrier 101 (e.g., resistance, capacitance, voltage decay, and ionic current flow). A signal source 108 can apply a voltage signal between working electrode 106 and counter electrode 107.

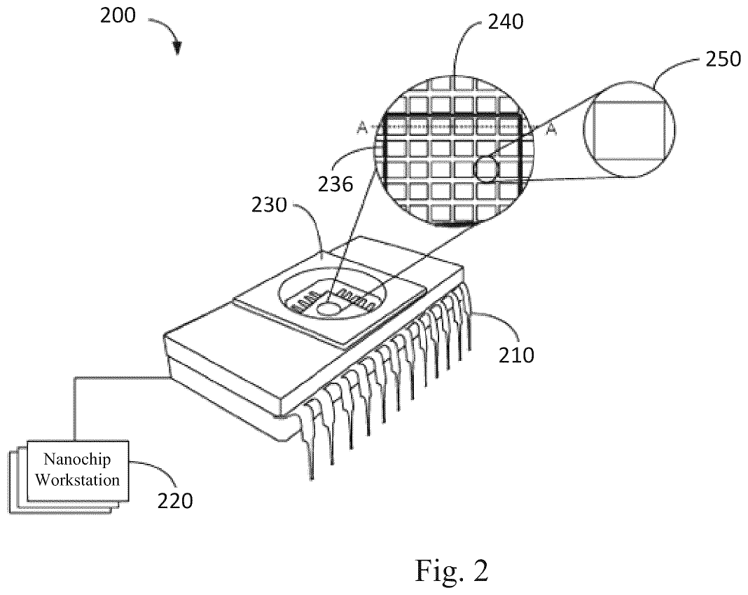

FIG. 2 is a top view of an exemplary embodiment of a nanopore sensor chip 200 having an array 240 of nanopore cells 250, each nanopore cell comprising a single nanopore sequencing complex 100. Each nanopore cell 250 may include a control circuit integrated on a silicon substrate of nanopore sensor chip 200. In some embodiments, side walls 236 are included in array 240 to separate groups of nanopore cells 250 so that each group can receive a different sample for characterization. Each nanopore cell can be used to sequence a nucleic acid. In some embodiments, nanopore sensor chip 200 includes a cover plate 230. In some embodiments, nanopore sensor chip 200 also includes a plurality of pins 210 for interfacing with other circuits, such as a computer processor.

In some embodiments, nanopore sensor chip 200 includes multiple chips in a same package, such as, for example, a Multi-Chip Module (MCM) or System-in-Package (SiP). The chips can include, for example, a memory, a processor, a field-programmable gate array (FPGA), an application-specific integrated circuit (ASIC), data converters, a high-speed I/O interface, etc.

In some embodiments, nanopore sensor chip 200 is coupled to (e.g., docked to) a nanochip workstation 220, which can include various components for carrying out (e.g., automatically carrying out) various embodiments of the processes disclosed herein. These process can include, for example, analyte delivery mechanisms, such as pipettes for delivering lipid suspension or other membrane structure suspension, analyte solution, and/or other liquids, suspension or solids. The nanochip workstation components can further include robotic arms, one or more computer processors, and/or memory. A plurality of polynucleotides can be detected on array 240 of nanopore cells 250. In some embodiments, each nanopore cell 250 is individually addressable.

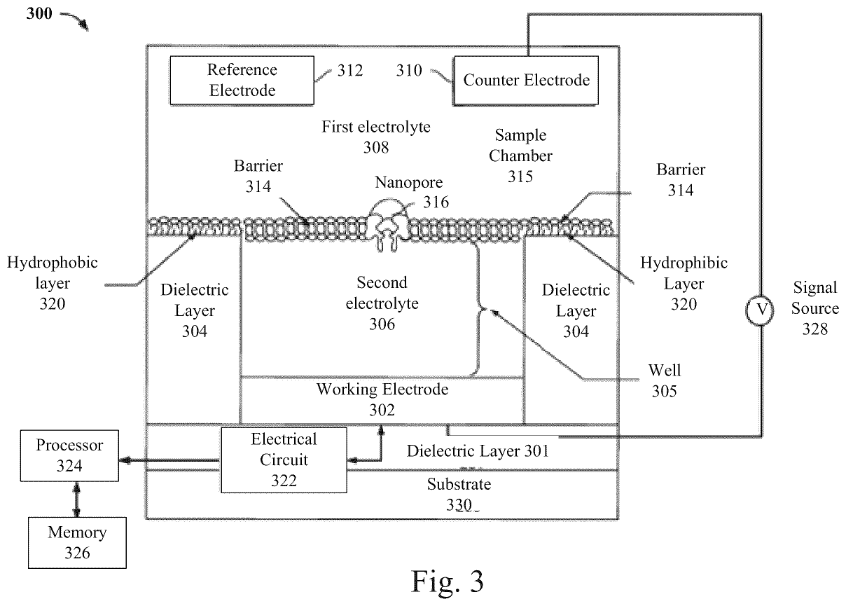

FIG. 3 illustrates an exemplary embodiment of a nanopore cell comprising a nanopore sequencing complex. Nanopore cell 300 can include a well 305 formed of dielectric layers 301 and 304; the barrier 314 formed over well 305; and a sample chamber 315 separated from well 305 by the barrier 314. Well 305 can contain a volume of the second electrolyte 306, and the sample chamber 315 can hold the first electrolyte 308 containing a nanopore, and the analyte of interest (e.g., a nucleic acid molecule to be sequenced). Nanopore cell 300 can include a working electrode 302 at the bottom of well 305 and a counter electrode 310 disposed in sample chamber 315. A signal source 328 can apply a voltage signal between working electrode 302 and counter electrode 310. A single nanopore can be inserted into barrier 314 by an electroporation process caused by the voltage signal, thereby forming a nanopore 316 in the barrier 314. The barrier (e.g., lipid bilayers 314 or other membrane structures) in the array can be neither chemically nor electrically connected to each other. Thus, each nanopore cell in the array can be an independent sequencing machine, producing data unique to the single polymer molecule associated with the nanopore that operates on the analyte of interest and modulates the ionic current through the otherwise impermeable barrier.

As shown in FIG. 3, nanopore cell 300 can be formed on a substrate 330, such as a silicon substrate. Dielectric layer 301 can be formed on substrate 330. Dielectric material used to form dielectric layer 301 can include, for example, glass, oxides, nitrides, and the like. An electric circuit 322 for controlling electrical stimulation and for processing the signal detected from nanopore cell 300 can be formed on substrate 330 and/or within dielectric layer 301. For example, a plurality of patterned metal layers (e.g., metal 1 to metal 6) can be formed in dielectric layer 301, and a plurality of active devices (e.g., transistors) can be fabricated on substrate 330. In some embodiments, signal source 328 is included as a part of electric circuit 322. Electric circuit 322 can include, for example, amplifiers, integrators, analog-to-digital converters, noise filters, feedback control logic, and/or various other components. Electric circuit 322 can be further coupled to a processor 324 that is coupled to a memory 326, where processor 324 can analyze the sequencing data to determine sequences of the polymer molecules that have been sequenced in the array.

Working electrode 302 can be formed on dielectric layer 301, and can form at least a part of the bottom of well 305.

Dielectric layer 304 can be formed above dielectric layer 301. Dielectric layer 304 forms the walls surrounding well 305. Dielectric material used to form dielectric layer 304 can include, for example, glass, oxide, silicon mononitride (SiN), polyimide, or other suitable hydrophobic insulating material. The top surface of dielectric layer 304 can be silanized. The silanization can form a hydrophobic layer 320 above the top surface of dielectric layer 304. In some embodiments, hydrophobic layer 320 has a thickness of about 1.5 nanometer (nm).

Well 305 formed by the dielectric layer walls 304 includes a second electrolyte 306 in contact with the working electrode 302. In some embodiments, second electrolyte 306 has a thickness of about three microns (μm).

The barrier 314 is formed on top of dielectric layer 304 and spanning across well 305. Barrier 314 is embedded with a single nanopore 316, which can be large enough for passing at least a portion of the analyte of interest and/or small ions (e.g., Na+, K+, Ca2+, Cl−) between the two sides of barrier 314. Sample chamber 315 is disposed on the cis side of barrier 314, and can hold a solution of the analyte of interest for characterization.

In some embodiments, various checks are made during creation of the nanopore cell as part of calibration. Once a nanopore cell is created, further calibration steps can be performed, e.g., to identify nanopore cells that are performing as desired (e.g., one nanopore in the cell). Such calibration checks can include physical checks, voltage calibration, open channel calibration, and identification of cells with a single nanopore.

In use, an active sequencing complex is generated at a plurality of nanopore sequencing complexes, a molecule enters into the channel of the nanopore to cause a change in one or more electrical characteristics of the nanopore sequencing complex, the changes are detected and transmitted to the computing system, and the computing system correlates the changes to the identity of the molecule(s) occupying the nanopore. In a SBS sequencing method, the molecule that enters the channel is a polymer tag of a tagged N5OP. In direct sequencing methods, the molecule that enters the channel is the nucleic acid of interest.

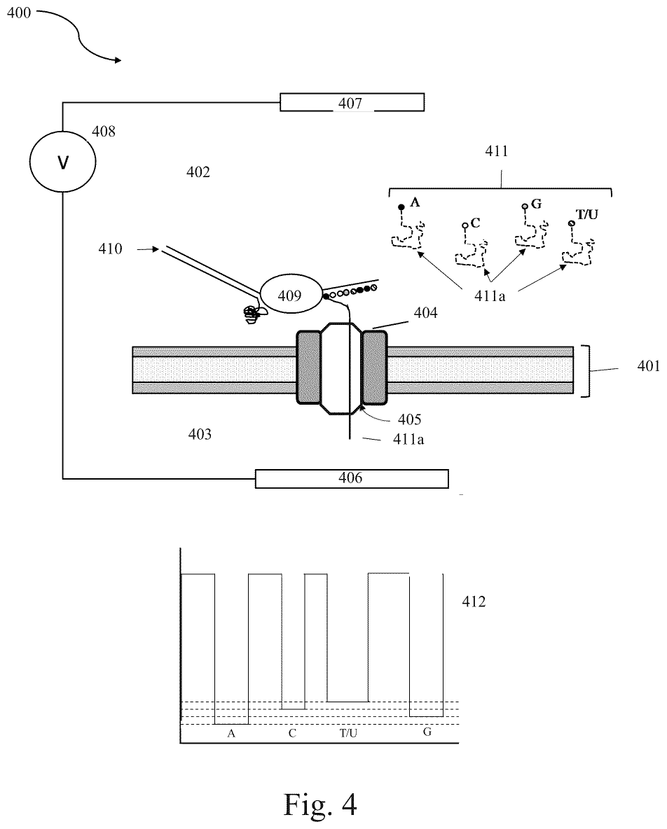

FIG. 4 illustrates an exemplary embodiment of an active sequencing complex 400 for performing a tag-based SBS nucleic acid sequencing. The electrically-resistive barrier 401 separates the first electrolyte solution 402 from the second electrolyte solution 403. The nanopore 404 is disposed in the electrically-resistive barrier 401, and the channel of the nanopore 405 provides a path through which ions can flow between the first electrolyte 402 and the second electrolyte 403. The working electrode 406 is disposed on the side of the electrically-resistive barrier 401 containing the second electrolyte 403 (termed the “trans side” of the electrically-resistive barrier) and positioned near the nanopore 404. The counter electrode 407 is positioned on the side of the electrically-resistive barrier 401 containing the first electrolyte 402 (termed the “cis side” of the electrically-resistive barrier). The signal source 408 is adapted to apply a voltage signal between the working electrode 406 and the counter electrode 407. A polymerase 409 is associated with nanopore 404, and a primed template nucleic acid 410 is associated with the polymerase 409. The first electrolyte 402 includes four different polymer-tagged nucleoside oligophosphates 411 (tag illustrated as 411a). The polymerase 409 catalyzes incorporation of the polymer-tagged nucleotides 411 into an amplicon of the template. When a polymer-tagged nucleoside oligophosphate 411 is correctly complexed with polymerase 409, the tag 411a can be pulled (e.g., loaded) into the nanopore by an electrical force, such as a force generated in the presence of an electric field generated by a voltage applied across the electrically-resistive barrier 401 and/or nanopore 404. While the tag 411a occupies the channel of the nanopore 404, it affects ionic flow through the nanopore 404, thereby generating an ionic blockade signal 412. Each nucleotide 411 has a unique polymer tag 411a that generates a unique ionic blockade signal due to the distinct chemical structure and/or size of the tag 411a. By identifying the unique ionic blockade signal 412, the identity of the unique tags 411a (and therefore, the nucleotide 410 with which it is associated) can be identified. This process is repeated iteratively with each nucleotide 411 incorporated into the amplicon. Exemplary tag-based SBS approaches and materials for performing such methods are described at, for example, WO 2012-083249, WO 2013/154999, US 2014/0309144, U.S. Pat. No. 9,017,937, WO 2015/148402, WO 2016/069806, WO 2016/144973, US 2016/0222363, US 2016/0333327, WO 2017/050728, WO 2017/184866, WO 2017/050722, US 2017/0267983, US 2018/0245147, US 2018/0094249, WO 2018/002125, and Kumar (each of which is incorporated herein by reference). Various tags have been proposed for use in such systems, including tags based on polypeptides (such as polylysine tags), polynucleotides, and polyethylene glycol. See, e.g., U.S. Pat. No. 8,652,779 and WO2017042038A1 (each of which is incorporated herein by reference).

### F. EXAMPLES

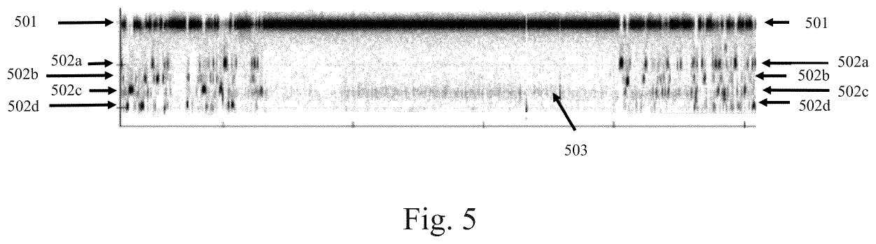

FIG. 5 illustrates a tag-based sequencing-by-synthesis (SBS) run using an α-hemolysin nanopore and negatively-charged tags. The dark band at the top is the open channel level 501 and a tag occupying the channel of the nanopore is recorded as a change in signal (in this case, conductance level) relative to open channel, with different tags resulting in different changes in signal 502a-502d. However, the present inventors have observed that a persistent background band is occasionally observed 503. The increased background results in convoluted tag signals and signal processing, which increases as the threading rate increases. This inherently limits the throughput and accuracy of tag-based SBS. Without being bound by theory, the aberrant pattern may result at least in part from threading of the negatively-charged template, primer, and/or amplicon nucleic acid through the positively-charged nanopore, and that the positive charge added to the nucleobase may reduce the attraction of between the template or primer and the nanopore.

### F1. Synthesis of 5-[3-(Trifluoroacetamino)-prop-1-ynyl]-2′-deoxycytidine-5′-O-triphosphate

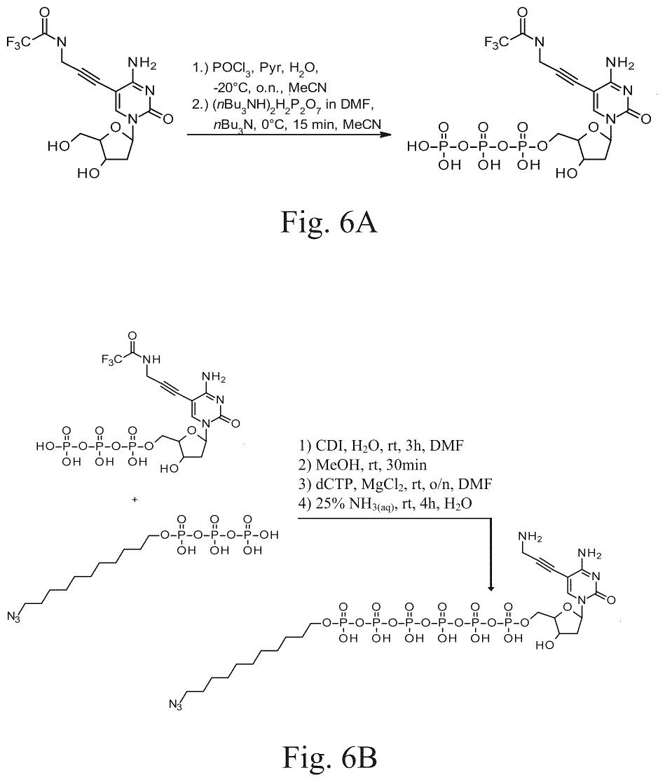

An exemplary synthesis scheme for obtaining a pyrimidine-containing N5OP having a PCM at the 5-position is illustrated at FIG. 6A.

83 μL of POCl3 were dissolved in 1 mL of dry MeCN and cooled to 0° C. 43 μL of pyridine and 9.6 μL of water were added and the solution was stirred for 30 min. 50 mg of nucleoside were dried under high vacuum for 4 hours and afterwards suspended in 1 mL of dry MeCN. Both solutions were cooled to −20° C. and then combined. The flask was sealed and the reaction kept at −20° C. overnight. The reaction was warmed to 0° C. A 0.5 M solution of tris(tetrabuylammonium) hydrogen pyrophosphate in dry dimethylformamide (DMF) and 316 μL tributylamine were added simultaneously and the reaction was stirred for 15 min. Afterwards the reaction was quenched with 5 mL of 0.2 M triethylamine acetate (TEAA) buffer pH=7 and stirred at 0° C. for 30 min The solvent was removed in vacuo and the crude product purified by reversed-phase HPLC (0.1 M TEAA/MeCN). Fractions containing the desired triphosphate were pooled and lyophilised. The product was obtained as an off-white solid. NMR and mass spectroscopy results are shown at Table 3:

TABLE 3

| | |
|---|---|
| ¹H NMR | δ ppm 8.17 (s, 1 H, H-6) 6.21 (t, J = 6.53 Hz, 1 H, |
| (400 MHz, D₂O) | H-1′) 4.56 (dt, J = 6.02, 3.64 Hz, 1 H, H-3′) |
| | 4.33 (s, 2 H, CH₂NH) 4.13-4.21 (m, 3 H, |
| | H-4′, H-5′) 2.40 (ddd, J = 14.09, 6.24, 4.14 Hz, |
| | 1 H, H-2′a) 2.26 (dt, J = 13.83, 6.70 Hz, 1 H, H-2′b) |
| MS (neg.-ESI) | 615.0 [M − H]⁻ |

### F2. Synthesis of P¹-[5-(3-Aminoprop-1-ynyl)-2′-deoxycytidine-5′]-P⁶-(11-azidoundecan-1-ol-1)hexaphosphate

A synthesis scheme for obtaining terminal phosphate-tagged pyrimidine-containing N5OP is illustrated at FIG. 6B.

A 5-substituted dNTP was obtained as illustrated at FIG. 6A. 39 mg of azidoundecanol triphosphate were dried under high vacuum for 3 h. Afterwards, 2 mL of dry DMF and 12.4 mg of carbonyldiimidazole (CDI) were added and the mixture was stirred at room temperature under argon for 3 h. In the meantime, 1 eq of dNTP and 12.2 mg of MgCl2 were dried under high vacuum. The reaction was quenched with 9 μL of MeOH and added to the dNTP. This mixture was kept at room temperature under argon over night. The reaction was diluted with 0.1 M TEAA buffer pH=8 and EDTA (119 mg) was added. After stirring at room temperature for 40 min the solvents were removed by lyophilisation. The resulting residue was taken up in 8 ml of 25% NH3(aq) and stirred at room temperature for 3 hours. The reaction was neutralized with 10% aqueous AcOH and solvents were removed by lyophilisation. Purification of the desired product was achieved by sequential ion exchange chromatography (DEAE-Sephadex; 25 mM Tris, 1 mM EDTA pH=8.5/A+1 M NaCl) and RP-HPLC (0.1 M TEAB pH=8/MeCN). Fractions containing product were identified by LC-MS, pooled and lyophilised. NMR and mass spectroscopy results are shown at Table 4:

TABLE 4

| | |
|---|---|
| ³¹P NMR | δ ppm −10.99-−10.73 (m, 1 P, P_{ζ}) −11.55 |
| (162 MHz, D₂O) | (d, J = 16.06 Hz, 1 P, P_{α}) −23.48-−22.79 |
| | (m, 4 P, P_{β}-P_{ε}) |
| MS (neg.-ESI) | 954.6 [M − H]⁻ |

### F3. Evaluation of Template Threading Behavior Using Tagged dN5OPs

The tagged dC5OP obtained in section C2 was used to evaluate the ability of bm-N5OPs to reduce threading behavior on a tag-based SBS system. A tagged bm-N5OP (the dC5OP obtained in section C2) was incorporated into a set of tagged N5OPs so that the resulting amplicon contains additional positive charge. It was theorized that the positive charge on the amplicon would neutralize at least a portion of the negative charge of the nucleic acids on the sequencing system, which would reduce the attraction of the nucleic acids to the positively charged alpha-hemolysin nanopore.

### Experimental Setup

The effect of the present bm-N5OPs on template threading phenomenon was evaluated using a nanopore array microchip essentially as described in US 2020/0216894 (incorporated herein by reference). The nanopore used in this case was a 6:1 αHL-derived nanopore, in which the “6” component consisted of polypeptides according to SEQ ID NO: 2, while the “1” component consisted of SEQ ID NO: 3 with a Pol6 derivative DNA-dependent DNA polymerase attached thereto via a Spy-Catcher/SpyTag attachment system. A 2.7 kb pUC plasmid was used as the template nucleic acid. Reference herein to “Template 1” or “Template 2” refer to the different strands of the plasmid. A potassium acetate electrolyte solution buffered to pH 7.8 with HEPES was used as the first and second electrolyte solutions. Two separate sets of terminally-phosphate tagged nucleotides were used:

TABLE 5
NATIVE dN5OP SET

| Nucleotide | Tag |
|---|---|
| dT6P | -TT-(sp2)₂₈-C₃ (SEQ ID NO: 4) |
| dG6P | -TT-(sp2)₁₂-dSp₁₀-(sp2)₆-C₃ (SEQ ID NO: 5) |
| dC6P | -(sp2)₁₇-(N3medT)₁₀-(sp2)₃-C₃ (SEQ ID NO: 6) |
| dA6P | -(sp2)₁₅-dCb₇-(sp2)₈-C₃ (SEQ ID NO: 7) |

TABLE 6
bm-dN5OP SET

| Nucleotide | Tag |
|---|---|
| dT6P | -TT-(sp2)₂₈-C₃ (SEQ ID NO: 4) |
| dG6P | -TT-(sp2)₁₂-dSp₁₀-(sp2)₆-C₃ (SEQ ID NO: 5) |
| bm-dC6P | -(sp2)₁₇-(N3medT)₁₀-(sp2)₃-C₃ (SEQ ID NO: 6) |
| dA6P | -(sp2)₁₅-dCb₇-(sp2)₈-C₃ (SEQ ID NO: 7) |

In each case, the “-” at the left of the tag indicates the end of the tag proximate to the attachment to the terminal phosphate of the nucleotide. When used in tags, “T” is deoxythymidine, “C” is deoxycytidine, “sp2” is an abasic site having the structure

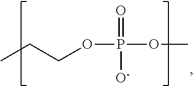

dCb is 5-methyl-deoxycytidine brancher phosphoramidite, and “N3medT” is N3-methyl-deoxythymidine.

### Effect of bm-N5OPs on Threading Behavior

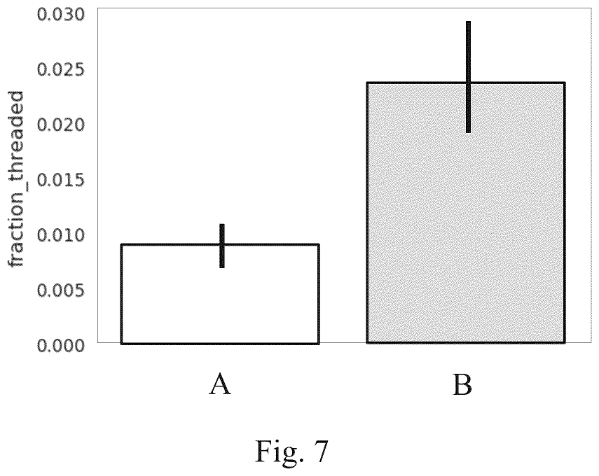

The fraction of pores exhibiting a threaded state was determined for each run. Results are shown at FIG. 7, with the Native dN5OP set labelled with “A” and the bm-dN5OP set labelled with “B.” A smaller fraction of pores demonstrated a threaded state with the bm-dN5OP set than with the native dN5OP set.

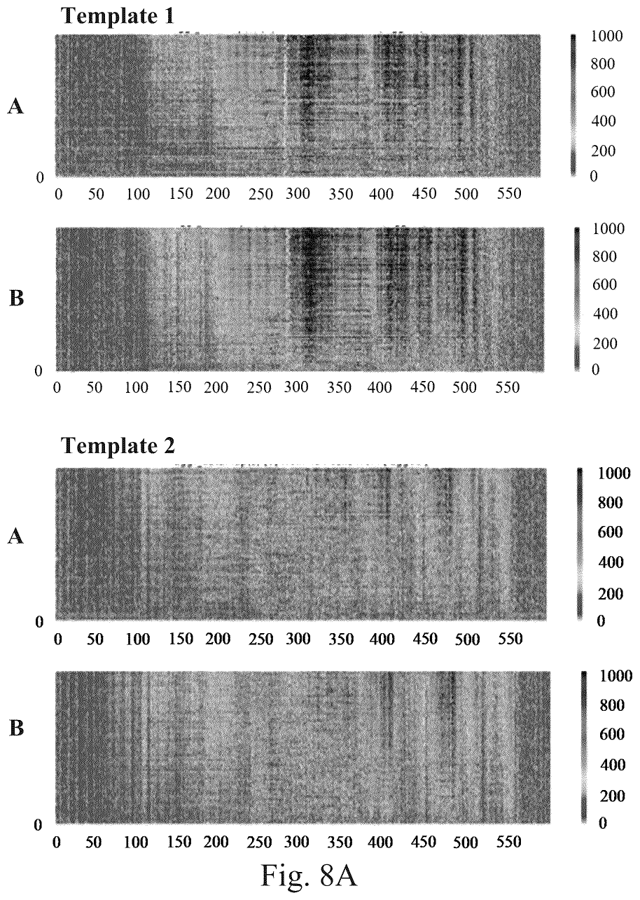

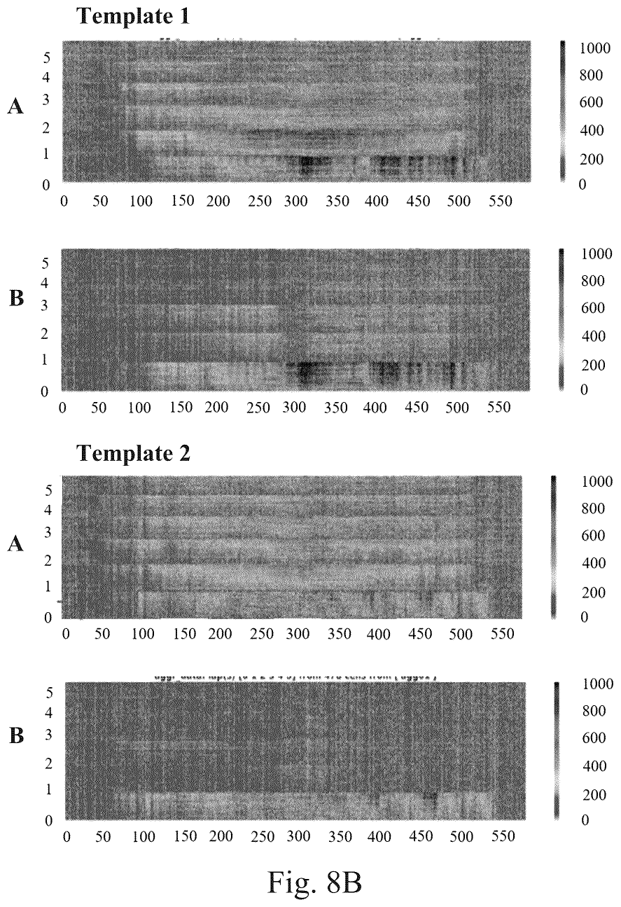

Additionally, a heat map of background template capture rate was also calculated for each experiment. FIG. 8A illustrates the background template capture rate for the first sequencing lap for each N5OP set and FIG. 8B illustrates the template capture rate for 5 total laps of sequencing. Heat maps labelled with “A” in FIGS. 8A and 8B were with the native dN5OP set. Heat maps labelled with “B” in FIGS. 8A and 8B were generated with the bm-dN5OP. As illustrated at FIG. 8A, no significant difference in background template capture rate was observed during the first lap of sequencing, which likely was due to insufficient charge neutralization during early amplicon production. At this point, the amplicon likely contained very few modified nucleobases relative to the negative charge of the template nucleic acids. After the second lap of sequencing, a considerable decrease in template background was observed when using the base-modified C5OP relative to the native C5OP. This is likely explained by the significant additional positive charge that has accumulated after 2 rounds of amplicon formation.

### Effect of bm-N5OPs on Nucleotide Deletion Profiles

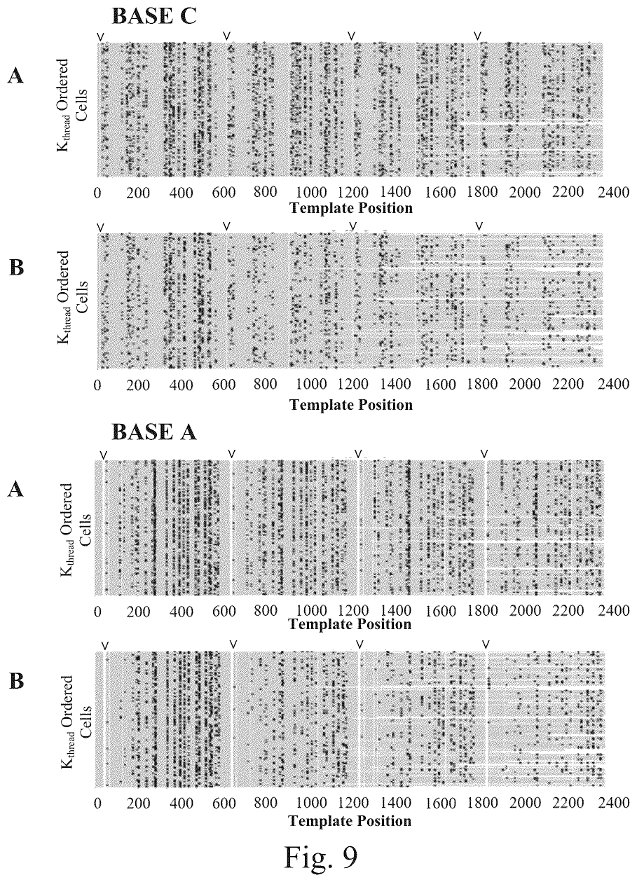

The tag levels of deoxycytidine and deoxyadenosine in the tag sets used in these examples were the closest to the background levels detected on the chip, making these the most likely nucleotides to be miscounted due to high background. It was therefore postulated that using the bm-dN5OP set would reduce the rate of A- and C-deletion relative to native tagged nucleotides. To test this, the rate of A- and C-deletion was calculated for each tag set. Results are shown at FIG. 9. Traces with the native dN5OP set are labelled with “A” and traces with the bm-dN5OP set are labelled with “B.” The X-axis is the position along the template at which a capture event is recorded and the Y-axis is the different cells of the chip. Black “V” marks at the top of each trace indicate the start of a pass along the template. Each instance of a C-deletion or an A-deletion is recorded as a black mark in the cell in which it was recorded. A reduction in deletions in passes 2, 3, and 4 was observed for both A and C when the bm-dN5OP set was used.

### G. REFERENCES

Cismas & Gimisis, exo-N-[2-(4-Azido-2,3,5,6-tetrafluorobenzamido)ethyl]-dC: a novel intermediate in the synthesis of dCTP derivatives for photoaffinity labelling, Tetrahedron Letters, 2008, Vol. 49, Issue 8, pp. 1336-1339.

Hocek & Fojta, Nucleobase modification as redox DNA labelling for electrochemical detection, Chemical Society Reviews, 2011, Vol. 40, Issue 12, pp. 5802-14.

Kumar et al., PEG-Labeled Nucleotides and Nanopore Detection for Single Molecule DNA Sequencing by Synthesis, Scientific Reports, 2012, Vol. 2, Issue 684; DOI: 10.1038/srep00684.

Xu et al., Fluorescent nucleobases as tools for studying DNA and RNA, Nature Chemistry, 2017, Vol. 9, Issue 11, pp. 1043-55.

### SPECIFICALLY INCLUDED EMBODIMENTS

The following embodiments are specifically contemplated as part of the disclosure. This is not intended to be an exhaustive listing of potentially claimed embodiments included within the scope of the disclosure.

Embodiment 1. A base-modified nucleoside-5′-oligophosphate (bmN5OP) or a salt thereof, the bmN5OP having a structure according to Formula 1:

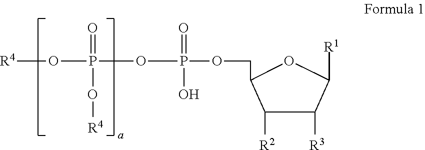

wherein: R¹ is selected from the group consisting of:

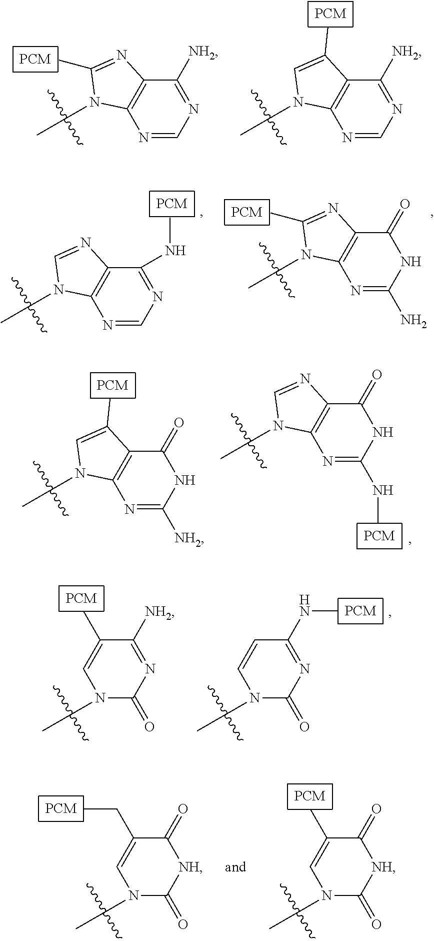

wherein: PCM is a moiety having a net-positive charge at 25° C. when in a reference solution buffered at pH 7-8 and comprising 450 mM potassium acetate; R² is selected from the group consisting of H and OH; R³ is selected from the group consisting of H, OH, F, and —O—CH₃; R⁴ is H or a nanopore-detectable tag construct, with the proviso that not more than one instance of R⁴ is the nanopore-detectable tag construct; and a is from 2 to 12.

Embodiment 2. The bmN5OP of embodiment 1, wherein PCM has a structure according to Formula 2:

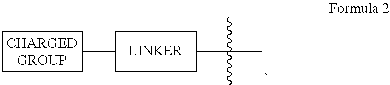

wherein CHARGED GROUP is a chemical group that has a net positive charge and LINKER is a chemical group covalently linking CHARGED GROUP to the nucleobase.

Embodiment 3. The bmN5OP of embodiment 2, wherein LINKER is selected from the group consisting of an alkane, an alkene, an alkyne, an aryl group, a heteroaryl group, an amide, an ether, and a polyether.

Embodiment 4. The bmN5OP of embodiment 3, wherein PCM is a structure selected from the group consisting of:

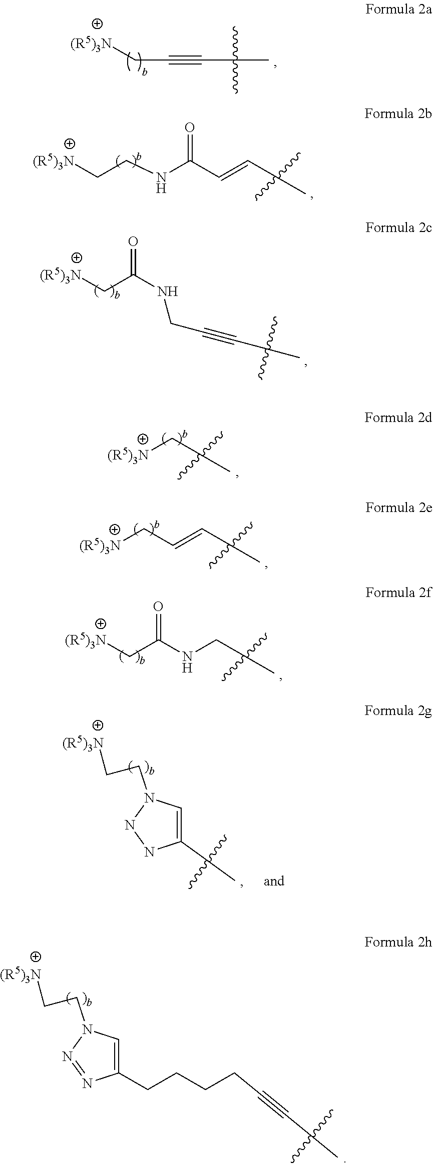

wherein: R⁵ is selected from the group consisting of H, F, Cl, Br, alkyl, and alkyl halide, and b is from 1 to 12.

Embodiment 5. A set of nucleoside-5′-oliogophosphates (N5OP) comprising: a deoxyadenosine 5′oliogophosphate (dA5OP); a deoxycytidine-5′-oliogophosphate (dC5OP); a deoxyguanosine-5′-oliogophosphate (dG5OP); and a deoxythymidine-5′-oliogophosphate (dT5OP) and/or a deoxyuridine-5′-oliogophosphate (dU5OP); wherein at least 1 of dA5OP, dC5OP, dG5OP, and dT5OP and/or dU5OP is the bmN5OP of any of embodiments 1-4.

Embodiment 6. The set of N5OPs of embodiment 5, wherein each of dA5OP, dT5OP or dU5OP, dC5OP, and dG5OP is the bmN5OP of any of embodiments 1-4.

Embodiment 7. The set of N5OPs of embodiments 5 or 6 wherein: dA5OP comprises a first nanopore-detectable tag construct, dC5OP comprises a second nanopore-detectable tag construct, dG5OP comprises a third nanopore-detectable tag construct, and dT5OP or dU5OP comprises a fourth nanopore-detectable tag construct, wherein the first, second, third, and fourth nanopore-detectable tag constructs are different from each other.

Embodiment 8. A method of obtaining a deoxyribonucleic acid (DNA) molecule, the method comprising polymerizing the set of N5OPs according to any of embodiments 5-7 in the presence of a template nucleic acid and an enzyme capable of polymerizing the N5OPs in a template-dependent manner

Embodiment 9. A method of sequencing a deoxyribonucleic acid (DNA) molecule, the method comprising: (a) generating an active sequencing complex on a nanopore-based sequencing platform, the active sequencing complex comprising: (a1) a sensing electrode; (a2) a nanopore positioned in proximity to the sensing electrode such that the sensing electrode can detect changes in at least one electrical characteristic of the nanopore; (a3) a DNA-dependent DNA polymerase linked to the nanopore; and (a4) a sequencing solution comprising the set of N5OPs according to claim 7; (b) incorporating an N5OP of the set of N5OPs into an amplicon of the DNA molecule in a template-dependent amplification reaction mediated by the DNA-dependent DNA polymerase using the DNA molecule as a template, wherein the nanopore-detectable tag construct of the N5OP incorporated into the amplicon inserts into the nanopore during incorporation, thereby changing the electrical characteristic of the nanopore detected by the sensing electrode; and (c) correlating the change in the electrical characteristic of the nanopore to the identity of the N5OP incorporated into the amplicon; and (d) repeating (a)-(c) for each N5OP incorporated into the amplicon, thereby sequencing the DNA molecule.

Embodiment 10. A set of nucleoside-5′-oliogophosphates (N5OP) comprising: an adenosine-5′-oliogophosphate (rA5OP); an uridine-5′-oliogophosphate (rU5OP); a cytidine-5′-oliogophosphate (rC5OP); and a guanosine-5′-oliogophosphate (rG5OP); wherein at least 1 of rA5OP, rU5OP, rC5OP, rG5OP is the bmN5OP of any of embodiments 1-4.

Embodiment 11. The set of N5OPs of embodiment 10, wherein each of rA5OP, rU5OP, rC5OP, and rG5OP is the bmN5OP of any of embodiment 1-4.

Embodiment 12. The set of N5OPs of embodiment 11, wherein: rA5OP comprises a first nanopore-detectable tag construct, rU5OP comprises a second nanopore-detectable tag construct, rC5OP comprises a third nanopore-detectable tag construct, and rG5OP comprises a fourth nanopore-detectable tag construct, wherein the first, second, third, and fourth nanopore-detectable tag constructs are different from each other.

Embodiment 13. A method of obtaining a ribonucleic acid (RNA) molecule, the method comprising polymerizing the set of N5OPs according to any of embodiments 10-12 in the presence of a template nucleic acid and an enzyme capable of polymerizing the N5OPs in a template-dependent manner.

Embodiment 14. A method of sequencing a ribonucleic acid (RNA) molecule, the method comprising: (a) generating an active sequencing complex on a nanopore-based sequencing platform, the active sequencing complex comprising: (a1) a sensing electrode; (a2) a nanopore positioned in proximity to the sensing electrode such that the sensing electrode can detect changes in at least one electrical characteristic of the nanopore; (a3) an RNA-dependent RNA polymerase linked to the nanopore; and (a4) a sequencing solution comprising the set of N5OPs according to claim 12; (b) incorporating an N5OP of the set of N5OPs into an amplicon of the RNA molecule in a template-dependent amplification reaction mediated by the RNA-dependent RNA polymerase using the RNA molecule as a template, wherein the nanopore-detectable tag construct of the N5OP incorporated into the amplicon inserts into the nanopore during incorporation, thereby changing the electrical characteristic of the nanopore detected by the sensing electrode; and (c) correlating the change in the electrical characteristic of the nanopore to the identity of the N5OP incorporated into the amplicon; and (d) repeating (a)-(c) for each N5OP incorporated into the amplicon, thereby sequencing the RNA molecule.

Embodiment 15. A nucleic acid, wherein at least 25% of nucleobases of the nucleic acid have a structure selected from the group consisting of:

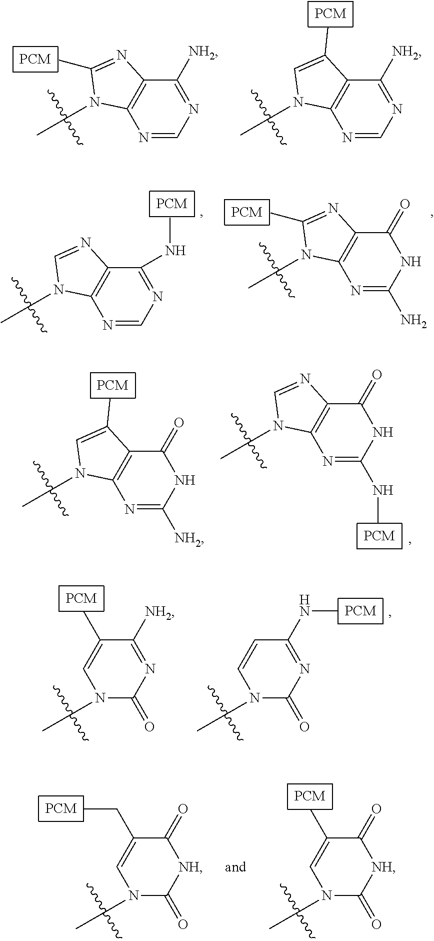

wherein PCM is a moiety having a net-positive charge at 25° C. when in a reference solution buffered at pH 7-8 and comprising 450 mM potassium acetate.

Embodiment 16. Use of the bmN5OP according to any of embodiments 1-4 for amplifying a template nucleic acid in a template-dependent manner or sequencing a template nucleic acid in a template-dependent manner on a nanopore-based sequencing system.

1. A base-modified nucleoside-5′-oligophosphate (bmN5OP) or a salt thereof, the bmN5OP having a structure according to Formula 1: [symbol]
Formula 1
wherein:
R¹ is selected from the group consisting of: [symbol]
, [symbol]
, [symbol]
, [symbol]
, [symbol]
, and
wherein:
PCM is a moiety having a net-positive charge at 25° C. when in a reference solution buffered at pH 7-8 and comprising 450 mM potassium acetate;
R² is selected from the group consisting of H and OH;
R³ is selected from the group consisting of H, OH, F, and —O—CH₃;
R⁴ is H or a nanopore-detectable tag construct, with the proviso that not more than one instance of R⁴ is the nanopore-detectable tag construct; and
a is from 2 to 12.

2. The bmN5OP of claim 1, wherein PCM has a structure according to Formula 2: [symbol]
Formula 2,
wherein CHARGED GROUP is a chemical group that has a net positive charge and LINKER is a chemical group covalently linking CHARGED GROUP to the nucleobase.

3. The bmN5OP of claim 2, wherein LINKER is selected from the group consisting of an alkane, an alkene, an alkyne, an aryl group, a heteroaryl group, an amide, an ether, and a polyether.

4. The bmN5OP of claim 3, wherein PCM is a structure selected from the group consisting of: [symbol]
Formula 2a, [symbol]
Formula 2b, [symbol]
Formula 2c, [symbol]
Formula 2d, [symbol]
Formula 2e, [symbol]
Formula 2f, [symbol]
Formula 2g, and [symbol]
Formula 2h,
wherein:
R⁵ is selected from the group consisting of H, F, Cl, Br, alkyl, and alkyl halide, and
b is from 1 to 12.

5. A set of nucleoside-5′-oliogophosphates (N5OP) comprising:
a deoxyadenosine-5′-oliogophosphate (dA5OP);
a deoxycytidine-5′-oliogophosphate (dC5OP);
a deoxyguanosine-5′-oliogophosphate (dG5OP); and
a deoxythymidine-5′-oliogophosphate (dT5OP) and/or a deoxyuridine-5′-oliogophosphate (dU5OP);
wherein at least 1 of dA5OP, dC5OP, dG5OP, and dT5OP and/or dU5OP is the bmN5OP of claim 1.

6. The set of N5OPs of claim 5, wherein each of dA5OP, dT5OP or dU5OP, dC5OP, and dG5OP is the bmN5OP of claim 1.

7. The set of N5OPs of claim 5 wherein:
dA5OP comprises a first nanopore-detectable tag construct,
dC5OP comprises a second nanopore-detectable tag construct,
dG5OP comprises a third nanopore-detectable tag construct, and
dT5OP or dU5OP comprises a fourth nanopore-detectable tag construct,
wherein the first, second, third, and fourth nanopore-detectable tag constructs are different from each other.

8. A method of obtaining a deoxyribonucleic acid (DNA) molecule, the method comprising polymerizing the set of N5OPs according claim 1 in the presence of a template nucleic acid and an enzyme capable of polymerizing the N5OPs in a template-dependent manner

9. A method of sequencing a deoxyribonucleic acid (DNA) molecule, the method comprising:
(a) generating an active sequencing complex on a nanopore-based sequencing platform, the active sequencing complex comprising:
(a1) a sensing electrode;
(a2) a nanopore positioned in proximity to the sensing electrode such that the sensing electrode can detect changes in at least one electrical characteristic of the nanopore;
(a3) a DNA-dependent DNA polymerase linked to the nanopore; and
(a4) a sequencing solution comprising the set of N5OPs according to claim 7;
(b) incorporating an N5OP of the set of N5OPs into an amplicon of the DNA molecule in a template-dependent amplification reaction mediated by the DNA-dependent DNA polymerase using the DNA molecule as a template, wherein the nanopore-detectable tag construct of the N5OP incorporated into the amplicon inserts into the nanopore during incorporation, thereby changing the electrical characteristic of the nanopore detected by the sensing electrode; and
(c) correlating the change in the electrical characteristic of the nanopore to the identity of the N5OP incorporated into the amplicon; and
(d) repeating (a)-(c) for each N5OP incorporated into the amplicon, thereby sequencing the DNA molecule.


SEQUENCE LISTING (7 sequences)

SEQ ID NO: 1 - protein, 305 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTNGLSAWSHPQFEK

SEQ ID NO: 2 - protein, 305 aa, synthetic construct
  REGION 1..305: note = aHL variant for "6" subunit
ADSDINIKTGTTDIGSNTTVKTGDLVTYDKENGMGKKVFYSFIDDKKHNKKLLVIRTKGTIAGQYRVYSEEGANKSGLAWPSAFKVQLQLPDNEVAQISDYYPRNSIDTKNYASTLTYGFNGNVTGGKGGGIGGLIGANVSIGATLNYKQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTNGLSAWSHPQFEK

SEQ ID NO: 3 - protein, 305 aa, synthetic construct
  REGION 1..305: note = aHL variant for "1" component
ADSDINIKTGTTDIGSNTTVKTGDLVTYDKENGMGKKVFYSFIDDKNHNKKLLVIRTKGTIAGQYRVYSEEGANKSGLAWPSAFKVQLQLPDNEVAQISDYYPRNSIDTKNYASTLTYGFNGNVTGGGGGGIGGLIGANVSIGHTLNYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTNGLSAWSHPQFEK

SEQ ID NO: 4-7 - skipped (4 entries)